(19) United States
(12) Patent Application Publication
(10) Pub. No.: US 2021/0024879 A1
(43) Pub. Date: Jan. 28, 2021

(54) BIOSYNTHETIC ACTIVITY OF THE ANAPLASMA PHAGOCYTOPHILUM AND EHRLICHIA CHAFFEENSIS PHAGOSOME IN A HOST CELL-FREE MEDIUM

(71) Applicant: Kansas State University Research Foundation, Manhattan, KS (US)

(72) Inventor: Roman R. Ganta, Manhattan, KS (US)

(21) Appl. No.: 15/733,731

(22) PCT Filed: Apr. 9, 2019
(86) PCT No.: PCT/US2019/026662

§ 371 Date: Oct. 7, 2020

Related U.S. Application Data
(60) Provisional application No. 62/654,965, filed on Apr. 9, 2018.

(51) Int. Cl. C12N 1/20

(52) U.S. Cl. CPC C12N 1/20; C12N 2500/32; C12N 2500/42; C12N 2500/34; C12N 2500/60

(57) ABSTRACT

Axenic media and methods for growing E. Chaffeensis and/or A. phagocytophilum are provided. In general, the axenic media includes intracellular phosphate buffer (IPB), a carbon source, FBS, a mixture of amino acids, and at least one further component selected from the group consisting of glucose 6-phosphate (G6P), ATP, DTT, GTP, UTP, CTP, and any combination thereof.

### BACKGROUND

Anaplasma phagocytophilum is an obligate Gram-negative, intracellular bacterium that causes an acute febrile illness known as anaplasmosis or human granulocytic anaplasmosis (HGA). HGA is an important cause of morbidity and is the second most common tick-transmitted disease in the United States. Disease activity has also been reported in Northern Europe and Southeast Asia. A. phagocytophilum is a member of the family Anaplasmataceae, which contains other tick-transmissible pathogens that infect peripheral white and red blood cells. This pathogen also causes infections and diseases in dogs, horses and cattle. Other Anaplasmataceae pathogens include Ehrlichia chaffeensis, the causative agent of human monocytic ehrlichiosis, Ehrlichia ewingii, the agent of canine and human ewingii ehrlichiosis, Ehrlichia canis, ehrlichia ruminantium, the agent of Heartwater disease in domestic and wild ruminants and Anaplasma marginale, which infects bovine erythrocytes, and Anaplasma platys, the agent of cyclic thrombocytopenia in dogs.

A. phagocytophilum survives and propagates within the host cell and can evade neutrophil antimicrobial functions. The life cycle of A. phagocytophilum and E. chaffeensis involves a tick vector and a mammalian host. A. phagocytophilum transitions between a smaller electron dense-cored cell (DC), which has a dense nucleoid, and a larger, pleomorphic electron lucent reticulate cell (RC), which has a dispersed nucleoid. Only DCs were observed binding to and inducing uptake by host cells. Between 4 to 12 hours, internalized DCs had developed into the no-infected form RC, which had initiated replication. Analogous to A. phagocytophilum transitioning, E. chaffeensis transitions between DC and RC; the smaller dense-core cells (DCs) with dense nucleoid and the larger pleomorphic reticulate cells (RCs). DCs are considered the infectious form of the bacterium, which enter naïve host cells by phagocytosis, then transform to non-infectious RCs within a phagosome and replicate prior to retransforming and releasing as DCs from the cells.

E. chaffeensis is an obligate intracellular, tick-transmitted bacterium that is maintained in nature in a cycle involving at least one and perhaps several vertebrate reservoir hosts. Human infections with E. chaffeensis cause the disease human monocytic ehrlichiosis (HME), which is characterized by an acute onset of febrile illness that can progress to a fatal outcome, particularly in immune compromised individuals, elderly and children. People undergoing blood transfusions and organ transplantations are also at high risk in acquiring E. chaffeensis infections and HME. Knowledge of the biology and natural history of E. chaffeensis, and of the epidemiology, clinical features, and laboratory diagnosis has expanded considerably in the period since its discovery.

The life cycle of E. chaffeensis involves a tick vector and a mammalian host. In both mammalian and tick cells, E. chaffeensis transitions between two forms; smaller dense-core cells (DCs) with dense nucleoid and larger reticulate cells (RCs) having uniformly dispersed nucleoid filaments and ribosomes, sometimes forming long projections of the cell wall, protrusions of the cytoplasmic membrane into the periplasmic space, or budding protoplast fragments into the periplasmic space. DCs are the infectious form of the bacterium, which enter naive host cells by phagocytosis, then transform to non-infectious RCs within a phagosome and replicate prior to retransforming and releasing as DCs from the cells. To date, there is not an understanding of the detailed differences in proteins expressed in the two distinct forms of E. chaffeensis in vertebrate and tick cells and how the entire process is regulated.

The ability to grow obligate intracellular bacteria under axenic conditions represents a major advancement, as it will enable new paths of investigation, such as aiding the manipulation of the pathogenic organisms in the absence of host cells, clonal purification of bacterial mutants, and detailed biochemical and genetic studies. However, axenic growth methods require considerable optimization to adapt to each obligate pathogen of interest. Recently, we demonstrated that the axenic medium can support both protein and DNA biosynthesis in RC forms of E. chaffeensis in the absence of host cells. These improvements may be possible if the axenic media growth is assessed with purified, host cell-derived E. chaffeensis RC-containing phagosomes in place of purified RCs.

### SUMMARY OF THE DISCLOSURE

The present disclosure overcomes the problems inherent in the art including those described above.

In one aspect of the disclosure, an axenic medium is provided for the cell free growth of a member of the family Anaplasmataceae. In some forms, the axenic medium comprises intracellular phosphate buffer (IPB), a carbon source, FBS, a mixture of amino acids, and at least one further component selected from the group consisting of glucose 6-phosphate (G6P), ATP, DTT, GTP, UTP, CTP, and any combination thereof In some forms the IPB comprises a mixture of at least one phosphate, at least one gluconate, and at least one chloride. In some forms, the phosphate is selected from the group consisting of potassium phosphate, sodium phosphate, and combinations thereof. The at least one phosphate is generally included at a final concentration of at least 1 mM, more preferably at least 2, 3, 4, 5, 6, 7, 8, 9, 10, 11, 12, 13, 14, or 15 mM. In some forms, the medium comprises a mixture of potassium phosphate and sodium phosphate. One preferred medium comprises 3-5 mM potassium phosphate and 8-12 mM sodium phosphate. In some forms, the gluconate is potassium gluconate. In some forms, the gluconate is included at a final concentration between 70 mM and 150 mM. In some forms, the gluconate is included at 70, 80, 90, 100, 110, 120, 130, 140, or 150 mM. In some forms, the at least one chloride is selected from the group consisting of potassium chloride, magnesium chloride, and any combination thereof. Generally, the at least one chloride is included in the medium at a final concentration between 0.5 mM and 12 mM including 0.5, 0.6, 0.7, 0.8, 0.9, 1, 1.1, 1.2, 1.3, 1.4, 1.5, 2, 2.5, 3, 3.5, 4, 4.5, 5, 5.5, 6, 6.5, 7, 7.5, 8, 8.5, 9, 9.5, 10, 10.5, 11, 11.5, and 12 mM. In some forms, the carbon source is alpha ketoglutarate and/or sodium acetate. In some forms, the alpha ketoglutarate and/or sodium acetate each individually and respectively comprise 0.1, 0.2, 0.3, 0.4, 0.5, 0.6, 0.7, 0.8, 0.9, or 1 mM. In some forms, the FBS supplements the IPB. In some forms, the FBS is 0.5, 0.6. 0.7, 0.8, 0.9, 1, 1.1, 1.2, 1.3, 1.4, or 1.5%. In some forms, the mixture of amino acids is an equimolar mixture. In some forms, there are at least 5, 10, 15, 16, 17, 18, 19, or 20 different or distinct amino acids in the amino acid mixture. In some forms, the amino acid mixture comprises 5, 10, 15, 20, 25, 30, 35, 40, 45, or 50 μM. In some forms, the G6P comprises 0.25, 0.3, 0.35, 0.4, 0.45, 0.5, 0.55, 0.6, 0.65, 0.7, or 0.75 mM. In some forms, the In some forms, the GTP, UTP, and/or CTP each individually and respectively comprises 25, 30, 35, 40, 45, 50, 55, 60, 65, 70, or 75 μNI. In some forms, GTP, UTP, and CTP are all present in the medium. In other forms, any one or two of GTP, UTP, and CTP are present in the medium. In some forms, ATP is also included. In some forms, the ATP is included instead of G6P. When ATP is included, it may represent any concentration between 0.1 to 1.5 mM including 0.5, 0.6, 0.7, 0.8, 0.9, 1, 1.1, 1.2, 1.3, 1.4, or 1.5 mM. Additionally, when ATP is included, a quantity of DTT may also be included. In some forms, the DTT concentration may comprise between 0.1 to 1 mM including 0.1, 0.2, 0.3, 0.4, 0.5, 0.6, 0.7, 0.8, 0.9, or 1 mM. In some forms, phagosomes are also included. In some forms, mitochondria are also included. In some forms, both mitochondria and phagosomes are also included. In some forms, the medium further includes another nutrient necessary or conducive to growth for the bacterium. In some forms, the member of the Anaplasmataceae family is selected from the group consisting of Ehrlichia chaffeensis, Ehrlichia ewingii, Ehrlichia canis, Ehrlichia ruminantium, Ehrlichia muris euclarancis, Anaplasma marginale, Anaplasma phagocytophilum, and any combination thereof.

In another aspect of the disclosure, axenic culture or growth methods for a member of the family Anaplasmataceae using a medium previously described for C. trachomitis, referred to as the CIP-1 medium, with some modifications, are provided. In some forms, the medium is as described above. In some forms, the member of the family Anaplasmataceae includes or is selected from the group consisting of Ehrlichia chaffeensis, Ehrlichia ewingii, Ehrlichia canis, Ehrlichia ruminantium, Ehrlichia muris euclarancis, Anaplasma marginale, A. phagocytophilum, and any combination thereof. In some forms, the culturing is performed at a pH between 5 and 9, preferably at a neutral pH. In some forms, the culturing is performed at a temperature between 24° C. to 40° C. In some forms, a time period for the culturing or growth comprises at least 12, 24, 36, 48, 60, 72, 84, 96, 108, 120, 132, 144, 156, 168, 180, or more hours. In some forms, the time period for growth or culturing comprises at least 1 day, more preferably between 1 and 7 days. In general, the member of the family Anaplasmataceae is placed in contact with the axenic medium. In some forms, the member is surrounded by or submerged in the axenic medium. In some forms, the axenic medium comprises intracellular phosphate buffer (IPB), a carbon source, FBS, a mixture of amino acids, and at least one further component selected from the group consisting of glucose 6-phosphate (G6P), ATP, DTT, GTP, UTP, CTP, and any combination thereof. In some forms the IPB comprises a mixture of at least one phosphate, at least one gluconate, and at least one chloride. In some forms, the phosphate is selected from the group consisting of potassium phosphate, sodium phosphate, and combinations thereof. The at least one phosphate is generally included at a final concentration of at least 1 mM, more preferably at least 2, 3, 4, 5, 6, 7, 8, 9, 10, 11, 12, 13, 14, or 15 mM. In some forms, the medium comprises a mixture of potassium phosphate and sodium phosphate. One preferred medium comprises 3-5 mM potassium phosphate and 8-12 mM sodium phosphate. In some forms, the gluconate is potassium gluconate. In some forms, the gluconate is included at a final concentration between 70 mM and 150 mM. In some forms, the gluconate is included at 70, 80, 90, 100, 110, 120, 130, 140, or 150 mM. In some forms, the at least one chloride is selected from the group consisting of potassium chloride, magnesium chloride, and any combination thereof. Generally, the at least one chloride is included in the medium at a final concentration between 0.5 mM and 12 mM including 0.5, 0.6, 0.7, 0.8, 0.9, 1, 1.1, 1.2, 1.3, 1.4, 1.5, 2, 2.5, 3, 3.5, 4, 4.5, 5, 5.5, 6, 6.5, 7, 7.5, 8, 8.5, 9, 9.5, 10, 10.5, 11, 11.5, and 12 mM. In some forms, the carbon source is alpha ketoglutarate and/or sodium acetate. In some forms, the alpha ketoglutarate and/or sodium acetate each individually and respectively comprise 0.1, 0.2, 0.3, 0.4, 0.5, 0.6, 0.7, 0.8, 0.9, or 1 mM. In some forms, the FBS supplements the IPB. In some forms, the FBS is 0.5, 0.6. 0.7, 0.8, 0.9, 1, 1.1, 1.2, 1.3, 1.4, or 1.5%. In some forms, the mixture of amino acids is an equimolar mixture. In some forms, there are at least 5, 10, 15, 16, 17, 18, 19, or 20 different or distinct amino acids in the amino acid mixture. In some forms, the amino acid mixture comprises 5, 10, 15, 20, 25, 30, 35, 40, 45, or 50 μNI. In some forms, the G6P comprises 0.25, 0.3, 0.35, 0.4, 0.45, 0.5, 0.55, 0.6, 0.65, 0.7, or 0.75 mM. In some forms, the In some forms, the GTP, UTP, and/or CTP each individually and respectively comprises 25, 30, 35, 40, 45, 50, 55, 60, 65, 70, or 75 μNI. In some forms, GTP, UTP, and CTP are all present in the medium. In other forms, any one or two of GTP, UTP, and CTP are present in the medium. In some forms, ATP is also included. In some forms, the ATP is included instead of G6P. When ATP is included, it may represent any concentration between 0.1 to 1.5 mM including 0.5, 0.6, 0.7, 0.8, 0.9, 1, 1.1, 1.2, 1.3, 1.4, or 1.5 mM. Additionally, when ATP is included, a quantity of DTT may also be included. In some forms, the DTT concentration may comprise between 0.1 to 1 mM including 0.1, 0.2, 0.3, 0.4, 0.5, 0.6, 0.7, 0.8, 0.9, or 1 mM. In some forms, phagosomes are also included. In some forms, mitochondria are also included. In some forms, both mitochondria and phagosomes are also included. In some forms, the medium further includes another nutrient necessary or conducive to growth for the bacterium.

In another aspect of the disclosure, the medium previously described for C. trachomatis, referred to as CIP-120, was modified to develop axenic culture or growth methods for RC-containing phagosomes from a member of the family Anaplasmataceae in the absence of host cells. In some forms, the medium is as described above. In some forms, the member of the family Anaplasmataceae includes Ehrlichia chaffeensis, Ehrlichia ewingii, Ehrlichia canis, Ehrlichia ruminantium, Ehrlichia muris euclarancis, Anaplasma marginale, A. phagocytophilum, and any combination thereof. In some forms, the culturing is performed at a pH between 5 and 9, preferably at a neutral pH. In some forms, the culturing is performed at a temperature between 24° C. to 40° C. In some forms, a time period for the culturing or growth comprises at least 12, 24, 36, 48, 60, 72, 84, 96, 108, 120, 132, 144, 156, 168, 180, or more hours. In some forms, the time period for growth or culturing comprises at least 1 day, more preferably between 1 and 7 days. In general, the member of the family Anaplasmataceae is placed in contact with the axenic medium. In some forms, the member is surrounded by or submerged in the axenic medium. In some forms, the axenic medium comprises intracellular phosphate buffer (IPB), a carbon source, FBS, a mixture of amino acids, and at least one further component selected from the group consisting of glucose 6-phosphate (G6P), ATP, DTT, GTP, UTP, CTP, and any combination thereof. In some forms the IPB comprises a mixture of at least one phosphate, at least one gluconate, and at least one chloride. In some forms, the phosphate is selected from the group consisting of potassium phosphate, sodium phosphate, and combinations thereof. The at least one phosphate is generally included at a final concentration of at least 1 mM, more preferably at least 2, 3, 4, 5, 6, 7, 8, 9, 10, 11, 12, 13, 14, or 15 mM. In some forms, the medium comprises a mixture of potassium phosphate and sodium phosphate. One preferred medium comprises 3-5 mM potassium phosphate and 8-12 mM sodium phosphate. In some forms, the gluconate is potassium gluconate. In some forms, the gluconate is included at a final concentration between 70 mM and 150 mM. In some forms, the gluconate is included at 70, 80, 90, 100, 110, 120, 130, 140, or 150 mM. In some forms, the at least one chloride is selected from the group consisting of potassium chloride, magnesium chloride, and any combination thereof. Generally, the at least one chloride is included in the medium at a final concentration between 0.5 mM and 12 mM including 0.5, 0.6, 0.7, 0.8, 0.9, 1, 1.1, 1.2, 1.3, 1.4, 1.5, 2, 2.5, 3, 3.5, 4, 4.5, 5, 5.5, 6, 6.5, 7, 7.5, 8, 8.5, 9, 9.5, 10, 10.5, 11, 11.5, and 12 mM. In some forms, the carbon source is alpha ketoglutarate and/or sodium acetate. In some forms, the alpha ketoglutarate and/or sodium acetate each individually and respectively comprise 0.1, 0.2, 0.3, 0.4, 0.5, 0.6, 0.7, 0.8, 0.9, or 1 mM. In some forms, the FBS supplements the IPB. In some forms, the FBS is 0.5, 0.6. 0.7, 0.8, 0.9, 1, 1.1, 1.2, 1.3, 1.4, or 1.5%. In some forms, the mixture of amino acids is an equimolar mixture. In some forms, there are at least 5, 10, 15, 16, 17, 18, 19, or 20 different or distinct amino acids in the amino acid mixture. In some forms, the amino acid mixture comprises 5, 10, 15, 20, 25, 30, 35, 40, 45, or 50 μNI. In some forms, the G6P comprises 0.25, 0.3, 0.35, 0.4, 0.45, 0.5, 0.55, 0.6, 0.65, 0.7, or 0.75 mM. In some forms, the In some forms, the GTP, UTP, and/or CTP each individually and respectively comprises 25, 30, 35, 40, 45, 50, 55, 60, 65, 70, or 75 μNI. In some forms, GTP, UTP, and CTP are all present in the medium. In other forms, any one or two of GTP, UTP, and CTP are present in the medium. In some forms, ATP is also included. In some forms, the ATP is included instead of G6P. When ATP is included, it may represent any concentration between 0.1 to 1.5 mM including 0.5, 0.6, 0.7, 0.8, 0.9, 1, 1.1, 1.2, 1.3, 1.4, or 1.5 mM. Additionally, when ATP is included, a quantity of DTT may also be included. In some forms, the DTT concentration may comprise between 0.1 to 1 mM including 0.1, 0.2, 0.3, 0.4, 0.5, 0.6, 0.7, 0.8, 0.9, or 1 mM. In some forms, phagosomes are also included. In some forms, mitochondria are also included. In some forms, both mitochondria and phagosomes are also included. In some forms, the medium further includes another nutrient necessary or conducive to growth for the bacterium.

In another aspect of the disclosure, a method of culturing or growing a member of the family Anaplasmataceae in an axenic medium is provided. In some forms, the member of the family Anaplasmataceae includes Ehrlichia chaffeensis, Ehrlichia ewingii, Ehrlichia canis, Ehrlichia ruminantium, Ehrlichia muris euclarancis, Anaplasma marginale, A. phagocytophilum, and any combination thereof. In general, the member of the family Anaplasmataceae is placed in contact with the axenic medium. In some forms, the member is surrounded by or submerged in the axenic medium. In some forms, the growth occurs at a pH between 5 and 9. In some forms, the growth occurs at a neutral pH. In some forms, the growth occurs at a temperature between 24° C. to 40° C. In some forms, a time period for the culturing or growth comprises at least 12, 24, 36, 48, 60, 72, 84, 96, 108, 120, 132, 144, 156, 168, 180, or more hours. In some forms, the time period for growth or culturing comprises at least 1 day, more preferably between 1 and 7 days. In some forms, the medium is as described above. In some forms, the axenic medium comprises intracellular phosphate buffer (IPB), a carbon source, FBS, a mixture of amino acids, and at least one further component selected from the group consisting of glucose 6-phosphate (G6P), ATP, DTT, GTP, UTP, CTP, and any combination thereof. In some forms the IPB comprises a mixture of at least one phosphate, at least one gluconate, and at least one chloride. In some forms, the phosphate is selected from the group consisting of potassium phosphate, sodium phosphate, and combinations thereof. The at least one phosphate is generally included at a final concentration of at least 1 mM, more preferably at least 2, 3, 4, 5, 6, 7, 8, 9, 10, 11, 12, 13, 14, or 15 mM. In some forms, the medium comprises a mixture of potassium phosphate and sodium phosphate. One preferred medium comprises 3-5 mM potassium phosphate and 8-12 mM sodium phosphate. In some forms, the gluconate is potassium gluconate. In some forms, the gluconate is included at a final concentration between 70 mM and 150 mM. In some forms, the gluconate is included at 70, 80, 90, 100, 110, 120, 130, 140, or 150 mM. In some forms, the at least one chloride is selected from the group consisting of potassium chloride, magnesium chloride, and any combination thereof. Generally, the at least one chloride is included in the medium at a final concentration between 0.5 mM and 12 mM including 0.5, 0.6, 0.7, 0.8, 0.9, 1, 1.1, 1.2, 1.3, 1.4, 1.5, 2, 2.5, 3, 3.5, 4, 4.5, 5, 5.5, 6, 6.5, 7, 7.5, 8, 8.5, 9, 9.5, 10, 10.5, 11, 11.5, and 12 mM. In some forms, the carbon source is alpha ketoglutarate and/or sodium acetate. In some forms, the alpha ketoglutarate and/or sodium acetate each individually and respectively comprise 0.1, 0.2, 0.3, 0.4, 0.5, 0.6, 0.7, 0.8, 0.9, or 1 mM. In some forms, the FBS supplements the IPB. In some forms, the FBS is 0.5, 0.6. 0.7, 0.8, 0.9, 1, 1.1, 1.2, 1.3, 1.4, or 1.5%. In some forms, the mixture of amino acids is an equimolar mixture. In some forms, there are at least 5, 10, 15, 16, 17, 18, 19, or 20 different or distinct amino acids in the amino acid mixture. In some forms, the amino acid mixture comprises 5, 10, 15, 20, 25, 30, 35, 40, 45, or 50 μNI. In some forms, the G6P comprises 0.25, 0.3, 0.35, 0.4, 0.45, 0.5, 0.55, 0.6, 0.65, 0.7, or 0.75 mM. In some forms, the In some forms, the GTP, UTP, and/or CTP each individually and respectively comprises 25, 30, 35, 40, 45, 50, 55, 60, 65, 70, or 75 μM. In some forms, GTP, UTP, and CTP are all present in the medium. In other forms, any one or two of GTP, UTP, and CTP are present in the medium. In some forms, ATP is also included. In some forms, the ATP is included instead of G6P. When ATP is included, it may represent any concentration between 0.1 to 1.5 mM including 0.5, 0.6, 0.7, 0.8, 0.9, 1, 1.1, 1.2, 1.3, 1.4, or 1.5 mM. Additionally, when ATP is included, a quantity of DTT may also be included. In some forms, the DTT concentration may comprise between 0.1 to 1 mM including 0.1, 0.2, 0.3, 0.4, 0.5, 0.6, 0.7, 0.8, 0.9, or 1 mM. In some forms, phagosomes are also included. In some forms, mitochondria are also included. In some forms, both mitochondria and phagosomes are also included. In some forms, the medium further includes another nutrient necessary or conducive to growth for the bacterium.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

### Color Drawings

The patent or application file contains at least one drawing executed in color. Copies of this patent or patent application publication with color drawing(s) will be provided by the Office upon request and payment of the necessary fee.

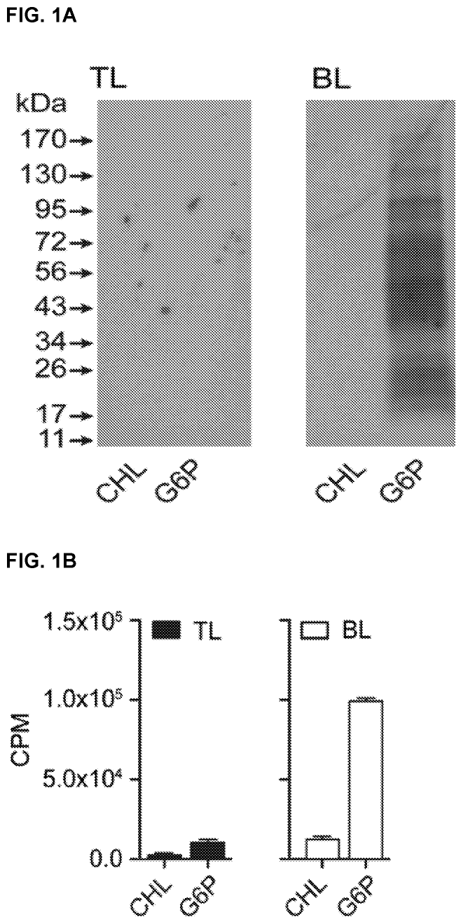

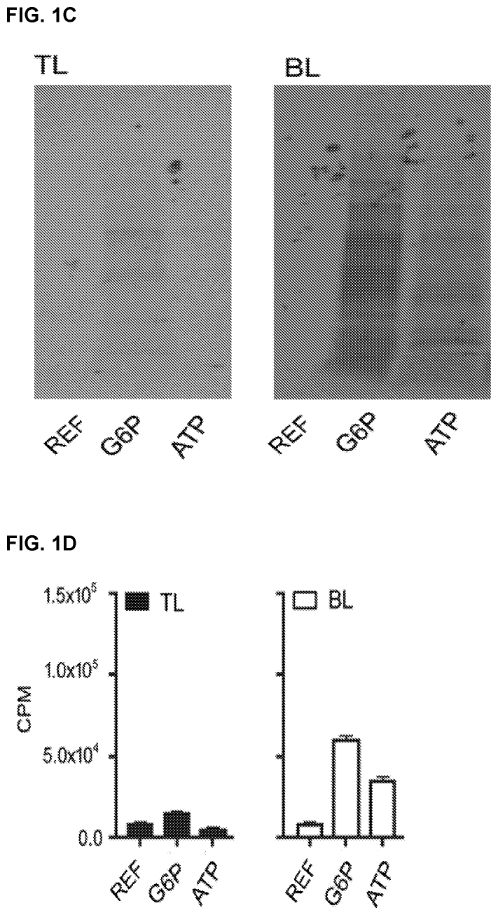

FIG. 1A illustrates E. chaffeensis organisms isolated from Vero cells used in the axenic media assessment using an autoradiography image to assess the incorporation of ³⁵S Cys-Met in E. chaffeensis recovered from the renografin centrifuged top (TL) and bottom (BL) layered fractions incubation in the axenic media with or without chloramphenicol (CHL) and with G6P as an energy source and incubated for 24 hours (G6P);

FIG. 1B is similar to FIG. 1A, but quantitation of radiolabel incorporation by scintillation count analysis data were presented for the ³⁵S Cys-Met incorporation into E. chaffeensis organisms;

FIG. 1C is similar to FIG. 1A except that the organisms were recovered from infected HL60 cells; this experiment also included a fraction of the purified organisms incubated in the axenic media with ATP as the energy source (ATP);

FIG. 1D is similar to FIG. 1B, but the scintillation counting data were presented;

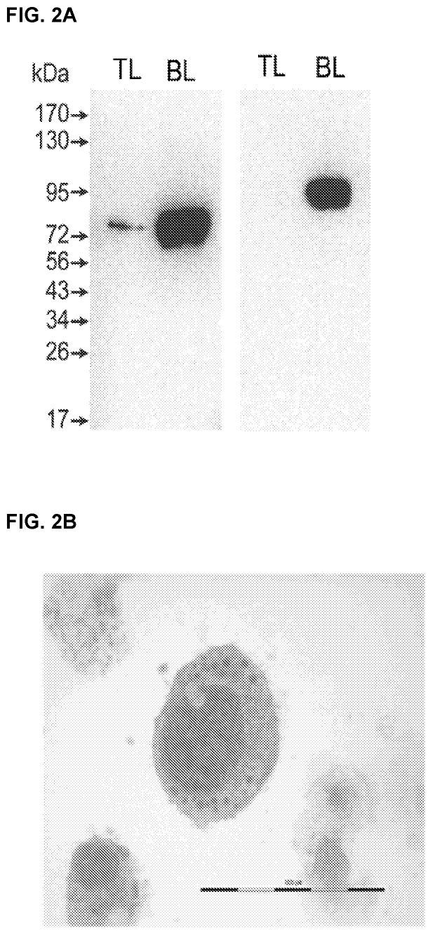

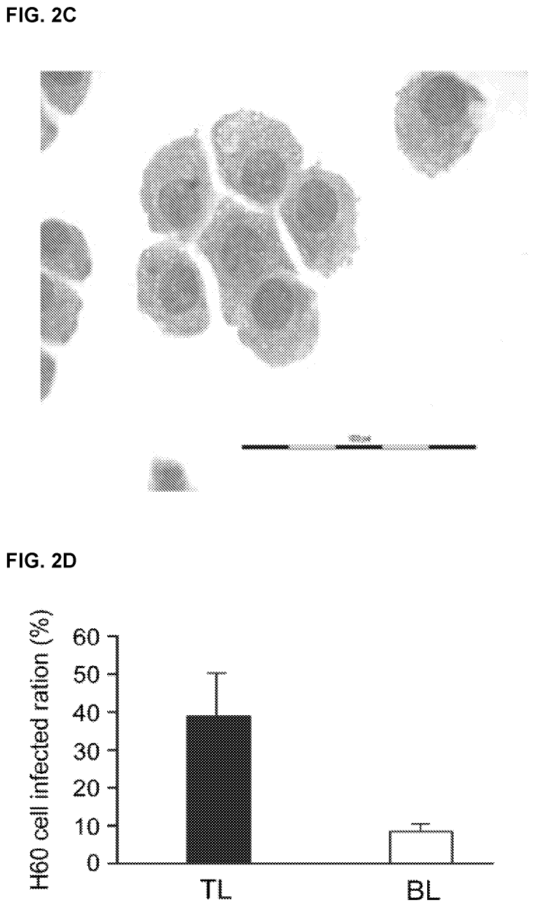

FIG. 2A is a Western blot analysis performed with DnaK antibody illustrating the presence of DCs and RCs assessed in the TL (which contains the infectious DC form) and BL (which contains the non-infectious RC form) of the renografin purified E. chaffeensis fractions which revealed higher levels of the protein expression in the BL derived bacterial fraction proteins (left half); similarly, ClpB protein expression was detected in the BL-derived proteins, but not in the top layer (right half);

FIG. 2B is a photograph illustrating cell-free E. chaffeensis recovered from TL (which contains the infectious DC form) and assessed for reinfection of naïve Vero cells; infection was detected only with the fraction derived from the TL;

FIG. 2C is a photograph illustrating cell-free E. chaffeensis recovered from BL (which contains the non-infectious RC form) and assessed for reinfection of naïve Vero cells; infection was not detected;

FIG. 2D is a graph illustrating the infectivity of fractionated Ehrlichia organisms from the TL (which contains the infectious DC form) and BL (which contains the non-infectious RC form) that was further confirmed by measuring the numbers of infected cells following incubation for 3 days following inoculation into naïve HL60 cultures;

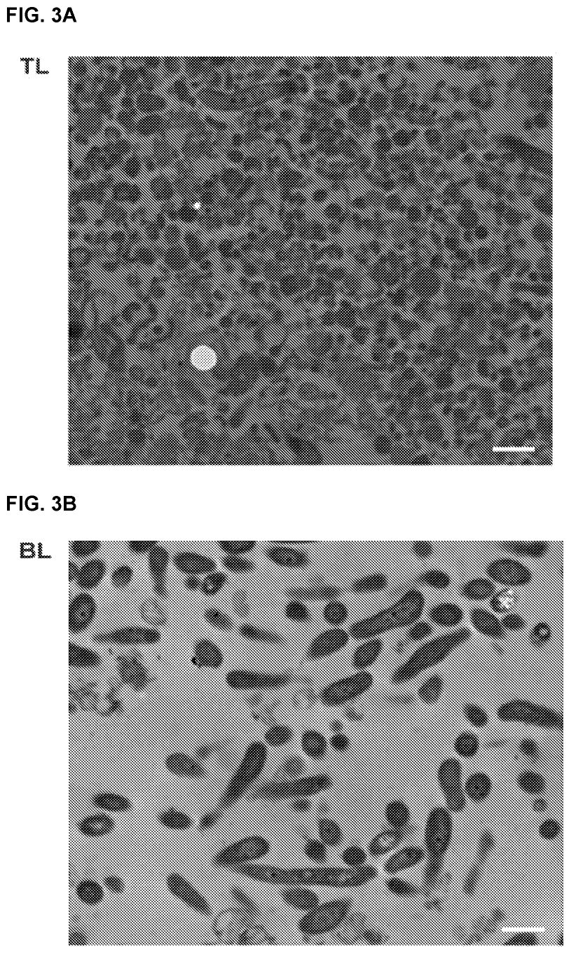

FIG. 3A is a photograph from transmission electron microscopy (TEM) analysis that was used to define the E. chaffeensis organisms present in the top layer (TL) (which contains the infectious DC form) of renografin fractions where prototypical small DCs and pleomorphic RCs of E. chaffeensis were observed;

FIG. 3B is a photograph from transmission electron microscopy (TEM) analysis that was used to define the E. chaffeensis organisms present in the bottom layer (BL) (which contains the non-infectious RC form) of renografin fractions where prototypical small DCs and pleomorphic RCs of E. chaffeensis were observed;

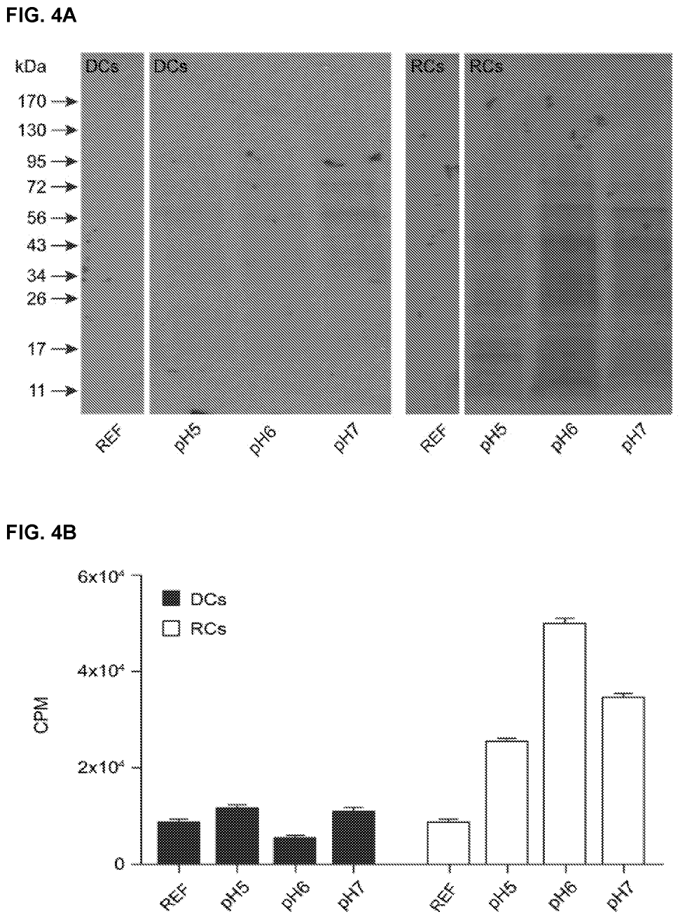

FIG. 4A is a set of photographs illustrating the impact of pH variations on the ³⁵S Cys-Met incorporation into E. chaffeensis cell-free organisms wherein autoradiography imaging was performed to assess the impact of three different pH units (5, 6 and 7 in the non-replicating infectious form, DC and the replication form, RC) . In the first lane, rifampin containing sample was included with media pH of 5.0 to serve as a negative control;

FIG. 4B is a graph illustrating the impact of pH variations on the ³⁵S Cys-Met incorporation into E. chaffeensis cell-free organisms wherein impact of three different pH units (5, 6 and 7 in the non-replicating infectious form, DC and the replication form, RC) was assessed and scintillation counting data were presented;

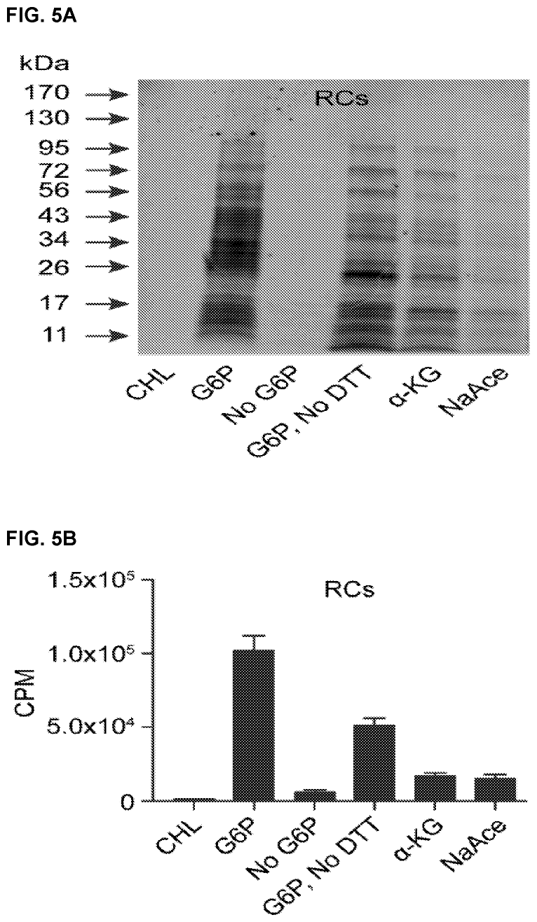

FIG. 5A is an autoradiograph image illustrating axenic media assessment using different carbon sources for the RC fraction the axenic media contained G6P with DTT (G6P); without an energy source (no G6P); with G6P in the absence of DTT (G6P, no DTT); with alpha keto glutaric acid (α-KG) or sodium acetate (NaAceA) as the energy sources. The first lane included axenic media with G6P and CHL to serve as the negative control;

FIG. 5B is a graph illustrating axenic media assessment using different carbon sources for the RC fraction the axenic media contained G6P with DTT (G6P); without an energy source (no G6P); with G6P in the absence of DTT (G6P, no DTT); with alpha keto glutaric acid (a-KG) or sodium acetate (NaAceA) as the energy sources and scintillation counting data were presented;

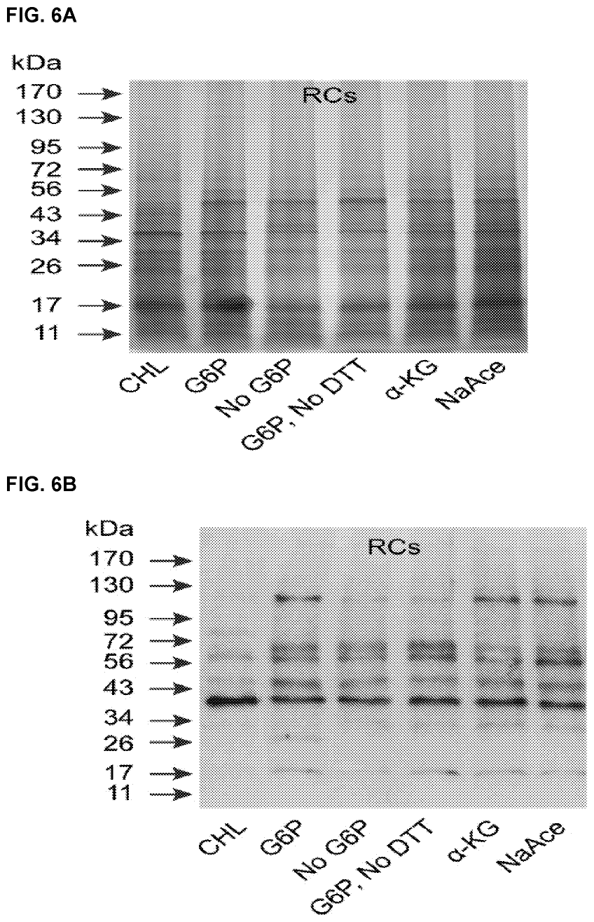

FIG. 6A is a photograph illustrating protein biosysthesis assessed by protein fractionation and Western blot analysis wherein silver stained PAGE in the presence of SDS resolved protein fractions were assessed for the protein abundance variations in RCs incubated in the axenic media with different carbon sources as in FIGS. 5A and 5B;

FIG. 6B is a photograph illustrating protein biosysthesis assessed by protein fractionation and Western blot analysis wherein silver stained PAGE in the presence of SDS resolved protein fractions were transferred to a nylon membrane and assessed by Western blot analysis using mouse polyclonal sera against E. chaffeensis;

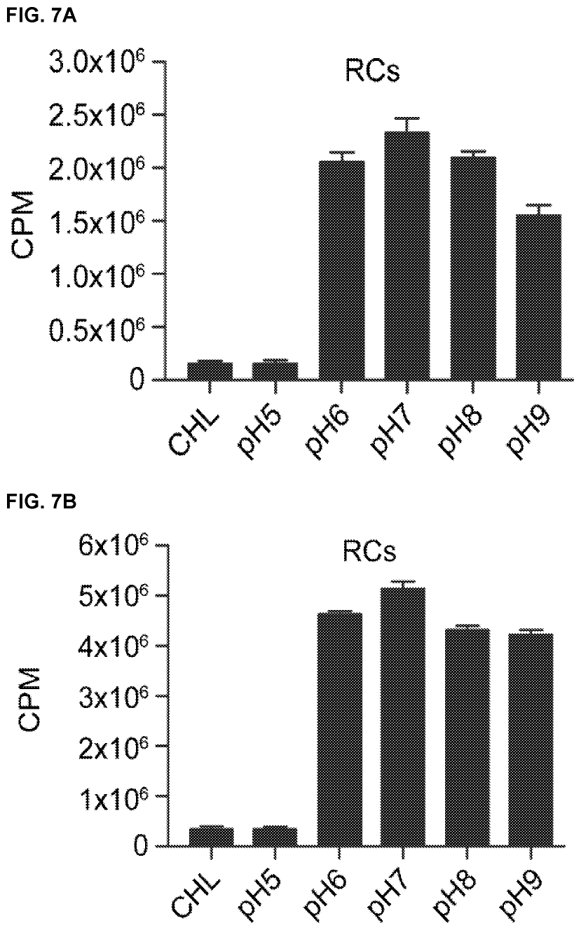

FIG. 7A is a graph illustrating protein biosynthesis and DNA synthesis assessed simultaneously for the RC form by measuring the ³⁵S Cys-Met in the axenic media at varying pHs of the media wherein scintillation counting data for the ³⁵S Cys-Met is assessed with G6P as the energy source;

FIG. 7B is a graph illustrating protein biosynthesis and DNA synthesis assessed simultaneously for the RC form by measuring the ³H thymidine incorporation in the axenic media at varying pHs of the media wherein panel B provides scintillation counting data for the ³H thymidine assessed with G6P as the energy source

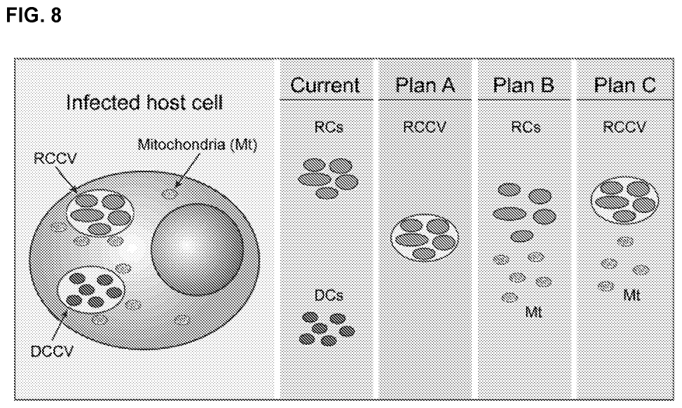

FIG. 8 is a schematic illustration of a proposed model to make improvements to the axenic media to promote E. chaffeensis replication in vitro in the absence of a host cell.

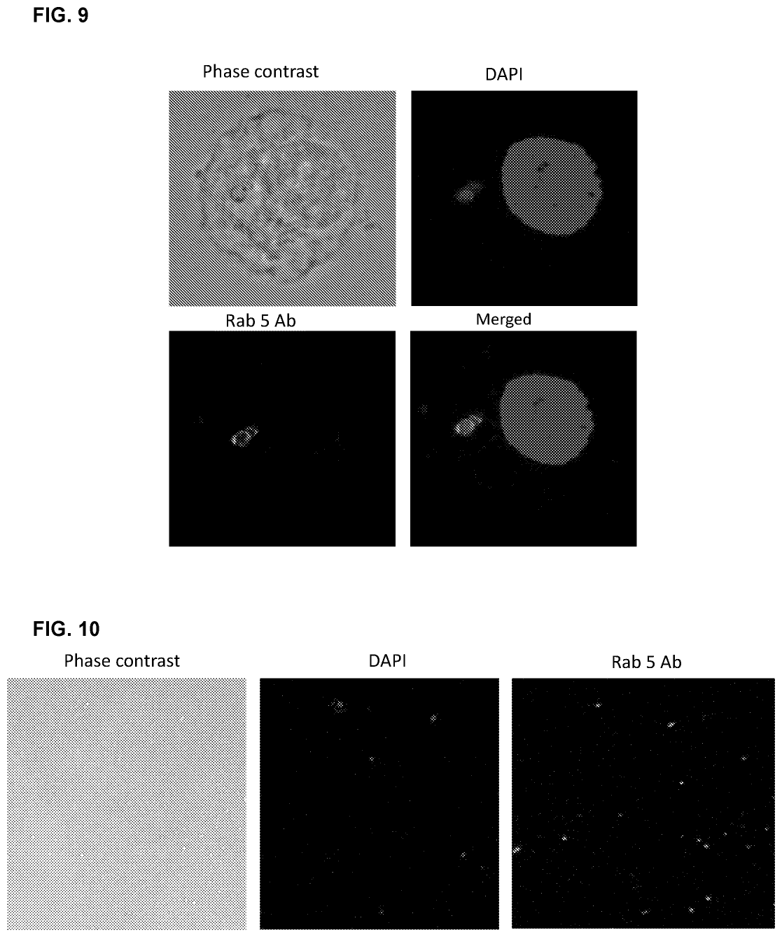

FIG. 9 is a set of representative confocal slices of Z-series, showing the phagosomes of the HL60 cell infected by A. phagocytophilum stained with DAPI and Rab 5 monoclonal antibody. The Z-stack images are of the 4^{th} slice of the total 7 slices;

FIG. 10 is a series of photographs illustrating the isolation of phagosomes from HL60 Cells infected by A. phagocytophilum with magnet assisted cell sorting;

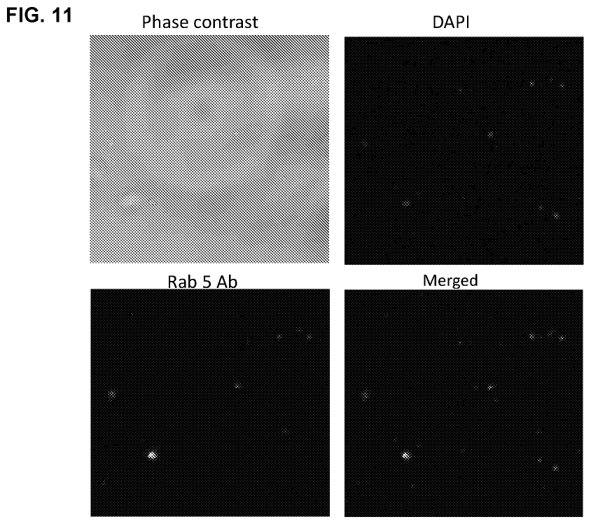

FIG. 11 is a series of photographs illustrating the isolation of phagosomes from HL60 cells infected by E. chaffeensis with magnet assisted cell sorting;

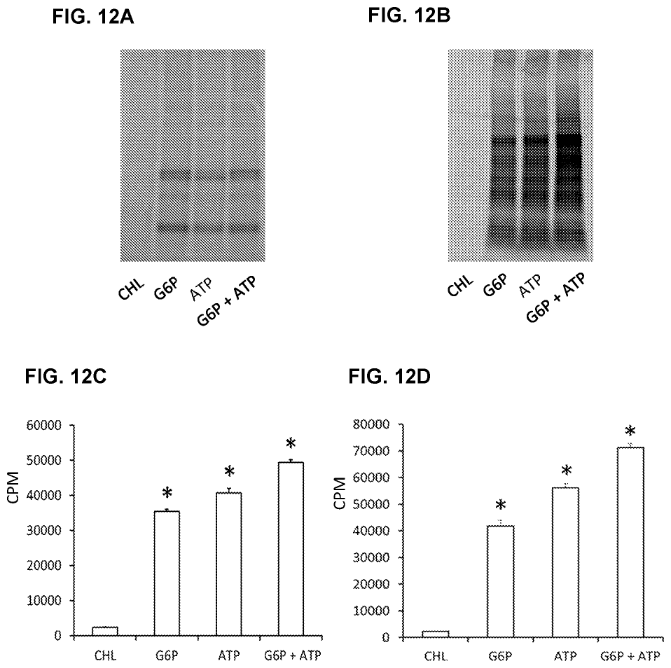

FIG. 12A is a photograph illustrating the impact of energy sources on incorporation of ^{S}35 Cys-Met into A. phagocytophilum cell-free phagosomes;

FIG. 12B is a photograph illustrating the impact of energy sources on incorporation of ^{S}35 Cys-Met into E. chaffeensis cell-free phagosomes;

FIG. 12C is a graph illustrating the impact of energy sources on incorporation of ^{S}35 Cys-Met into A. phagocytophilum cell-free phagosomes;

FIG. 12D is a graph illustrating the impact of energy sources on incorporation of ^{S}35 Cys-Met into E. chaffeensis cell-free phagosomes;

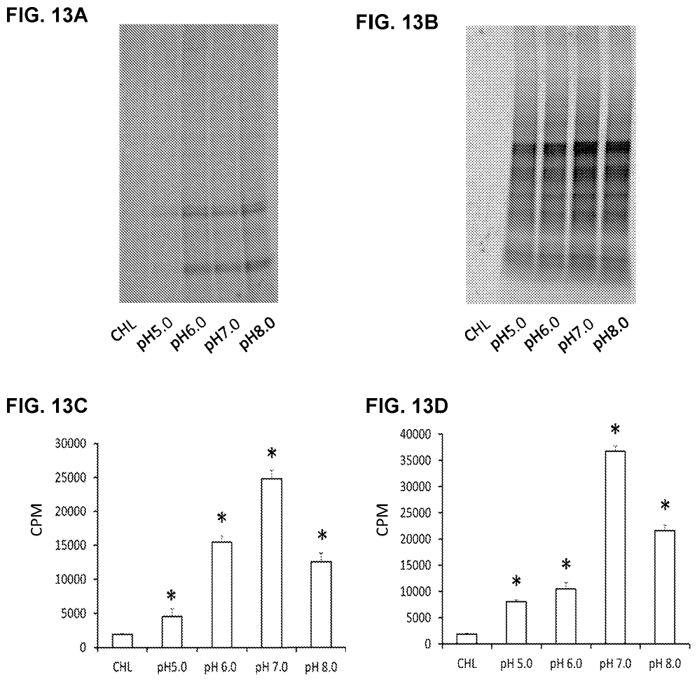

FIG. 13A is a photograph illustrating the impact of pH variations on incorporation of ^{S}35 Cys-Met into A. phagocytophilum cell-free phagosomes;

FIG. 13B is a photograph illustrating the impact of pH variations on incorporation of ^{S}35 Cys-Met into E. chaffeensis cell-free phagosomes;

FIG. 13C is a graph illustrating the impact of pH variations on incorporation of ^{S}35 Cys-Met into A. phagocytophilum cell-free phagosomes;

FIG. 13D is a graph illustrating the impact of pH variations on incorporation of ^{S}35 Cys-Met into E. chaffeensis cell-free phagosomes;

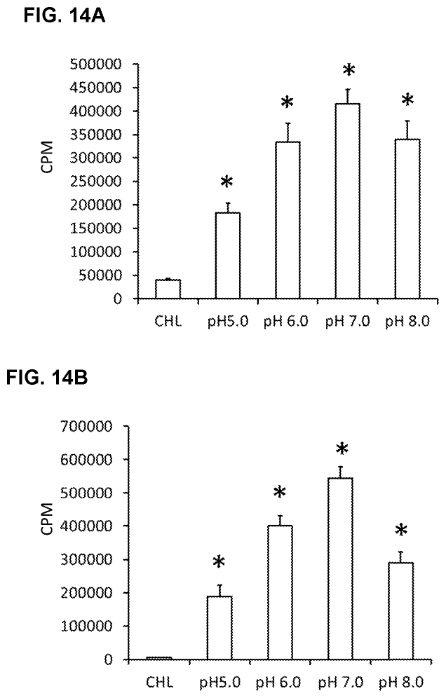

FIG. 14A is a graph illustrating DNA synthesis assessed simultaneously by measuring the 3H thymidine incorporation in the axenic media at varying pHs of the media in A. phagocytophilum cell-free phagosomes;

FIG. 14B is a graph illustrating DNA synthesis assessed simultaneously by measuring the 3H thymidine incorporation in the axenic media at varying pHs of the media in E. chaffeensis cell-free phagosome.

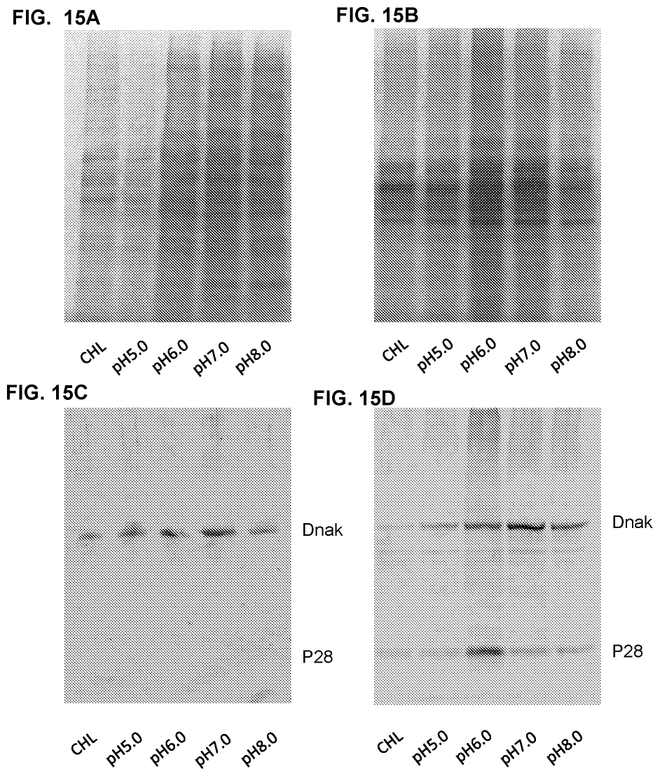

FIG. 15A is a photograph illustrating protein biosynthesis assessed by protein fractionation and Western blot analysis wherein silver stained SDS containing polyacrylamide gel-resolved protein fractions were assessed for the protein abundance variations in A. phagocytophilum cell-free phagosome;

FIG. 15B is a photograph illustrating protein biosynthesis assessed by protein fractionation and Western blot analysis wherein silver stained SDS containing polyacrylamide gel-resolved protein fractions were assessed for the protein abundance variations in E. chaffeensis cell-free phagosome;

FIG. 15C is similar to FIG. 15A but wherein protein biosynthesis was assessed by Western blot analysis using mouse monoclonal Antibody DnaK and P28 against A. phagocytophilum; and

FIG. 15D is similar to FIG. 15B but wherein protein biosynthesis was assessed by Western blot analysis using mouse monoclonal antibody DnaK and P28 against E. chaffeensis.

### DETAILED DESCRIPTION

The following detailed description and examples set forth preferred materials and procedures used in accordance with the present disclosure. It is to be understood, however, that this description and these examples are provided by way of illustration only, and nothing therein shall be deemed to be a limitation upon the overall scope of the present disclosure. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure unless noted otherwise. All parts and percents referred to herein are on a weight basis and all temperatures are expressed in degrees centigrade unless otherwise specified. Further unless noted otherwise, all components of the disclosure are understood to be disclosed to cover “comprising”, “consisting essentially of”, and “consisting of” claim language as those terms are commonly used in patent claims.

### EXAMPLE 1

This example evaluates the possibility of developing axenic culture methods for E. chaffeensis using a medium previously described for C. trachomitis, referred to as the CIP-1 medium. The results are described using the axenic medium for its value in supporting both protein and DNA biosynthesis in DC and RC forms of E. chaffeensis in the absence of host cells.

Materials and Methods

Cultivation of E. chaffeensis: E. chaffeensis was cultivated in canine macrophage cell line, DH82 as described previously. Similarly, E. chaffeensis in Vero cells (ATCC, Manasas, Va.) was cultured in the complete MEM medium (Gibco/ThermoFisher Scientific, Waltham, Mass.) supplemented with 7% fetal bovine serum (Invitrogen/ThermoFisher Scientific, Waltham, Mass.) and 2 mM L-glutamine (Mediatech, Manassas, Va.). Cultivation of E. chaffeensis in HL60 cells (ATCC, Manassas, Va.) in complete RPMI 1640 medium (Gibco/ThermoFisher Scientific) supplemented with 10% fetal bovine serum (Invitrogen/ThermoFisher Scientific, Waltham, Mass.) and 2 mM L-glutamine (Mediatech, Manassas, Va.), by following the protocols described elsewhere for Anaplasma phagocytophilum strain NCH-1. To prepare cell-free Ehrlichia inocula, about 80-100% E. chaffeensis-infected DH82 cells in a T25 flask were harvested by centrifugation at 400×g for 10 minutes at 4° C. The pellets were resuspended in 5 ml of serum-free medium, and the cells were disrupted with glass beads by vortexing twice for 30 seconds. The cell debris and unbroken cells were removed by centrifugation at 200×g for 10 minutes at 4° C. The supernatant was passed through a 2.7 μm pore-size syringe filter (Whatman, Pittsburgh, Pa.). HL60 cells were incubated with host cell-free E. chaffeensis (multiplicity of infection of 100:1, bacteria to host cell) for 120 minutes to allow for internalization. Non-ingested E. chaffeensis were removed by washing with PBS, and the cells were incubated for an additional 3 days in T150 flask. Similar infection protocol is followed when infecting Vero cells or DH82 cells. When the infectivity reached to 80-90%, the infected host cell cultures were harvested by centrifugation at 500×g for 5 minutes at 4° C. and used for purifying the host cell-free bacteria, as outlined below.

Purification of E. chaffeensis: E. chaffeensis organisms in the forms of dense-core cells (DCs) and reticulate cells (RCs) were purified by subjecting to renografin density gradient centrifugation as described previously with some minor modifications. In brief, pellets of infected host cells were suspended in sterile PBS. The cells were then homogenized at 4° C. using a 10 ml syringe with a 23^{½}-G needle; typically 10-15 strokes were used to disrupt the cells. Homogenization was carried out until approximately 90% of cells were disrupted without major breakage of nuclei, as monitored by light microscopy. The disrupted cell suspension was centrifuged at 500×g for 5 minutes at 4° C. The supernatant was collected and filtered through 2.7 μm sterile syringe filter. The filtered supernatant was then centrifuged at 15,000×g for 15 minutes at 4° C. The pellet was resuspended into sterile PBS and 2 mL of the suspension was layered over discontinuous renografin gradients (3 mL 25%, 4 mL of 35% renografin in PBS, vol/vol). These gradients were centrifuged at 100,000×g for 1 h at 4° C. using a Swinging Bucket rotor (S50-ST) in a Sorvall MTX150 ultracentrifuge (Waltham, Mass.). Fractions at the interfaces of PBS-25% and 25-35% renografin were collected using a sterile syringe, diluted with three volumes of PBS, and then centrifuged at 15,000×g for 15 minutes at 4° C. The pellets were washed with PBS to remove residual renografin by repeating the centrifugation step 15,000×g for 15 minutes at 4° C., and then the final purified pellets were resuspended in PBS for use in the cell free activity experiments.

Preparation of axenic medium: The axenic medium was prepared according to the previous study on C. trachomatis cultured in axenic medium, and the compositions and concentrations of each component in the axenic medium were listed in Table 1. Depending on the experiment carried out, the medium contained or excluded glucose 6-phoshate (G6P) or adenosine triphosphate (ATP) or alpha ketoglutarate or sodium acetate to serve as carbon sources. Similarly, pH of the media was modified as per the experimental need.

TABLE 1
CIP-1 medium recipe

| | | mg/100 | Final | |
|---|---|---|---|---|
| Component | Formula | ml | concentration | |
| 2X IPB^{a} | | | | |
| Potassium phosphate | KH₂PO₄ | 136 | 5 | mM |
| Sodium phosphate | Na₂HPO₄ × 7H₂O | 536 | 10 | mM |
| Potassium gluconate | C₆H₁₁KO₇ | 5140 | 110 | mM |
| Potassium chloride | KCl | 120 | 8 | mM |
| Magnesium chloride | MgCl₂ × 6H₂O | 40 | 1 | mM |
| Nutrients | | | | |
| Dithiothreitol | — | — | 0.5 | mM |
| Glucose-6-phosphate | — | — | 0.5 | mM |
| Adenosine triphosphate | — | — | 1 | mM |
| Fetal bovine serum | — | — | 1% | |
| 20 amino acids^{b} | — | — | 25 | μM |
| Cytidine 5′-triphosphate | — | — | 50 | μM |
| Guanosine 5′- | — | — | 50 | μM |
| triphosphate | | | | |
| Uridine 5′-triphosphate | — | — | 50 | μM |

^{a}Intracellular Phosphate Buffer. Adjust final volume of complete medium to 1X.
^{b}For radiolabeling, the concentration of cysteine and methionine are reduced to 1 μM. Medium pH adjusted to 7.2-3 using 6N potassium hydroxide prior to sterile filtration.

Protein synthesis by ³⁵S-cysteine-methionine incorporation: Protein synthesis in cell-free purified fractions of E. chaffeensis was measured by incorporation of ³⁵S-Cys-Met (Perkin Elmer, Waltham, Mass.) as described by Omsland, A., et al., in Developmental stage-specific metabolic and transcriptional activity of Chlamydia trachomatis in an axenic medium. Proc Natl Acad Sci USA. 109, 19781-19785 (2012), the teachings and content of which are hereby incorporated by reference. For normalization of bacterial total protein content, the suspension of E. chaffeensis cell-free fractions were lysed in 1% (wt/vol) SDS for 5 minutes at 100° C. and the total protein concentration was determined using Protein Assay kit (Bio-Rad, Hercules, Calif.). Subsequently, the suspensions of E. chaffeensis cell-free fractions were equally split into micro-centrifuge tubes at the amount of 30 μg total protein. Partially opened micro-centrifuge tubes containing 500 μL of medium supplemented with 70 μCi of ³⁵S-Cys-Met were incubated at 37° C. for 24 hours in a tri-gas incubator set to maintain 2.5% O₂. E. chaffeensis cell-free organisms were pelleted at the end of incubation by centrifugation at 15,000×g for 15 minutes at 4° C., washed with K-36 buffer (0.05M K₂HPO₄, 0.05M KH₂PO₄, 0.1M KCl, 0.15M NaCl, pH7.0) twice, and disrupted by adding 30 μL of 2×SDS-PAGE sample buffer and by boiling for 5 minutes. Ten μL of lysate each was then transferred to a tube containing 5 mL of biosafe liquid II and used for quantification of ³⁵S-Cys-Met incorporation using the protocol 4 (³⁵S) in a liquid scintillation counter (TRI-CARB 2100TR, PerkinElmer, Waltham, Mass.). For visualizing the radiolabel incorporation into bacterial proteins, equal volumes of sample lysates were also separated in an SDS/PAGE and the gel was dried and exposed to an X-ray film. Similarly, cell-free growth experiments were carried out in the absence of ³⁵S-Cys-Met, resolved on an SDS-PAGE gel, and stained using silver nitrate staining kit (Pierce/ThermoFisher Scientific) as per the manufacturer's recommendations.

DNA synthesis by ³H-thymidine incorporation: Purified E. chaffeensis cell-free fractions were also assessed for incorporation of ³H-thymidine (Perkin Elmer, Waltham, Mass.) into the bacterial DNA simultaneously with the incorporation ³⁵5-Cys-Met into proteins. Briefly, E. chaffeensis cell-free organisms were incubated for 48 hours at 37° C. with 2.5% O₂ in micro-centrifuge tubes containing 500 μL of medium supplemented with 20 μCi of ³H-thymidine and 70 μCi of ³⁵S-Cys-Met. E. chaffeensis were pelleted at 15,000×g for 15 minutes at 4° C., washed with K-36 twice, lysed in 30 μL of 2×SDS-PAGE sample buffer and then boiled for 5 min. 10 μL of lysate each was added into 5 mL of Biosafe liquid II (Grainger, Hartford, Conn.) and used for quantification of ³H-thymidine incorporation using the protocol 10 (³H) and ³⁵S-Cys-Met incorporation (the protocol 4, ³⁵S) by liquid scintillation counting (TRI-CARB 2100TR, PerkinElmer, Waltham, Mass.), respectively.

Sodium dodecyl sulfate polyacrylamide gel electrophoresis: Five μL of NuPAGE SDS sample buffer and 2 μL of NuPAGE reducing agent (Invitrogen/ThermoFisher Scientific) were added to each of 10 μL of sample solution following cell-free incubation experiments in the axenic medium, boiled for 5 min, and then loaded onto a Mini-PROTEAN Precast Bis-Tris 4% to 14% gels (Bio-rad, Hercules, Calif.) and subjected to electrophoresis (100 mA/gel for 60 minutes). The gels were then stained with Silver staining kit (Pierce/ThermoFisher Scientific) according to the manufacturer's recommendations.

Western blot analysis to assess protein synthesis: For detection of the DnaK and ClpB proteins of E. chaffeensis, the above described electrophoresed proteins were transferred onto a nitrocellulose membrane (Thermo Fisher Scientific, Waltham, Mass.) by subjecting to electro-blotting using an electrophoretic transfer unit (Bio-Rad). Protein transfer buffer was prepared as per the manufacturer's instructions and used in the protein transfer protocols. Subsequently, E. chaffeensis ClpB or DnaK expressions were assessed using the polyclonal rabbit antisera raised against a recombinant E. chaffeensis proteins for ClpB or DnaK, respectively. Secondary anti-rabbit antibody conjugated with horseradish peroxidase (Sigma-Aldrich, St. Louis, Mo., USA) and Super Signal West Pico Chemiluminescent Substrate (Thermo Fisher Scientific, Waltham, Mass., USA) were used for the signal detection, respectively.

Preparation of E. chaffeensis cultures for use in transmission electron microscopy: Purified E. chaffeensis DCs and RCs by renografin density gradient centrifugation were resuspended in PBS and used in transmission electron microscopy analysis by following the methods described previously. Briefly, all centrifugation steps used in preparing the TEM samples were performed at 4° C. for 5 minutes at 200×g, unless otherwise specified. The cultures in PBS were fixed with 1 ml of Karnovsky's fixative containing 2% paraformaldehyde, 2.5% gluteraldehyde in 0.1 M cacodylate buffer (pH7.4) overnight. The cell-free E. chaffeensis organisms were then washed three times with 1 ml of 0.1 M cacodylate buffer and were incubated in 1 ml of 1% osmium tetraoxide in 0.1 M cacodylate buffer for one hour, washed three times using deionized water and then resuspended in 2% trypsin soy agar solution. Each sample was diced with a teflon coated razor blade and placed in a wheaton glass vials with 50% ethanol at room temperature for 15 min, then stained with 70% ethanolluranyl acetate in the dark for one hour at room temperature. The bacterial suspension in soy agar was then subjected to dehydration process with an ethanol gradient of increased concentrations from 50% to 100%. All samples embedded in the soy agar-gluteraldehyde resin were subsequently transferred to silicon molds to allow for polymerization to be completed. All blocks were examined under a dissecting scope to identify a sample that was flush to the end of the block using an Ultracut E-Reichert-Jung ultramicrotome. Sections of 0.5 μm size were cut in the range of 75-90 nm, and transferred to Athene Thin Bar copper grids (Ted Pella, Redding, Calif.). The grids were stained with uranyl acetate in 70% ethanol followed by Reynold's lead citrate. The stained grids were examined under a Hitachi H-300 electron microscope (Hitachi High-Tech, San Jose, Calif.).

Results

Protein synthesis in cell-free replicating form of E. chaffeensis assessed in axenic media. The axenic medium used for C. trachomatis is a complex mixture containing intracellular phosphate buffer (IPB) supplemented with 1% FBS, 25 μM of equimolar mix of all 20 amino acids (AA), 0.5 mM glucose 6-phosphate (G6P) or 1.0 mM ATP with or without 0.5 mM DTT, and 50 μM each of GTP, UTP, and CTP as described in, except when using alpha ketoglutarate (0.5 mM) or sodium acetate (0.5 mM) as the carbon source. When assessing ³⁵S Cys-Met incorporation, concentration of these two cold amino acids were reduced to 1 μM and then the two radioactive amino acids were supplemented with 70 μCi of ³⁵S-Cys-Met. Protein synthesis in the axenic media is verified with inclusion of chloramphenicol (CHL) or rifampin (RIF), which serve as negative controls as protein synthesis would not be expected in the presence of these antibiotics. In the current study, we prepared the medium with or without an energy source and used it to determine if it supports E. chaffeensis protein synthesis in the absence of a host cell. E. chaffeensis dense-core cells (DCs) and reticulate cells (RCs) were purified from the infected Vero cells or DH82 cells by renografin gradient centrifugation which fractionated as PBS/25% renografin fraction (top layer) and 25-35% renografin fraction (bottom layer), respectively. Incubation of the purified Ehrlichia organisms promoted the protein synthesis in the bottom layer of the renografin fraction where the RCs appeared to have concentrated, while the protein synthesis was nearly absent or weaker in the top fraction, which is likely to contain DCs (FIG. 1). The protein synthesis was abolished when CHL was included in the media (FIG. 1) and similarly with the inclusion of rifampin.

In particular, FIGS. 1A, 1B, 1C, and 1D illustrate the incorporation of ³⁵S Cys-Met into E. chaffeensis organisms recovered from the bottom layer inter face of renografin fractionation when incubated in the axenic medium. Panel A illustrates E. chaffeensis organisms isolated from Vero cells used in the axenic media assessment; autoradiography image to assess the incorporation of ³⁵S Cys-Met in E. chaffeensis recovered from the renografin centrifuged top (TL) and bottom (BL) layered fractions incubation in the axenic media with or without chloramphenicol (CHL) and with G6P as an energy source and incubated for 24 h (G6P). FIG. 1B is similar to FIG. 1A but quantitation of radiolabel incorporation by scintillation count analysis data were presented for the ³⁵S Cys-Met incorporation into E. chaffeensis organisms. FIG. 1C is also similar to FIG. 1A except that the organisms were recovered from infected HL60 cells. This experiment also included a fraction of the purified organisms incubated in the axenic media with ATP as the energy source (ATP). FIG. 1D is similar to FIG. 1C, but the scintillation counting data were presented.

The presence of DCs and RCs in the top and bottom layers of the renografin-purified fractions was confirmed by multiple experiments; Western blot analysis, in vitro infection assessment (FIGS. 2A, 2B, 2C, and 2D) and by transmission electron microscopy (FIGS. 3A and 3B). We previously reported the enhanced expression of E. chaffeensis DnaK in RCs compared to the infectious DCs. In the current study, DnaK expression was assessed by Western blot analysis using the total proteins recovered from the E. chaffeensis organisms fractionated as the top and bottom layers on renografin gradient centrifugation (interfaces between PBS and 25% renografin fraction and 25% and 35% renografin fractions, respectively). DnaK expression was significantly higher in the bottom layer compared to that found in the top layer (FIG. 2A left half). Similarly, ClpB protein was detected in the bottom layer, but not in the top layer. (FIG. 2A, right half). The protein expression data for ClpB and DnaK suggest that the replicating form of E. chaffeensis is fractionated at the interface of the 25-35% renografin. To further confirm the presence of DCs and RCs in top and bottom layers, respectively, the cell-free organisms recovered from each of the two fractions were used to re-infect naive Vero cells (FIG. 2B). E. chaffeensis infection in Vero cells was primarily detected when fractionated organisms from the top layer were used, but not from the bottom layer. Infectivity of fractionated Ehrlichia organisms from the top renografin layer was also confirmed by judging their ability to infect HL60 cells where we estimated the percent of infected host cells (FIG. 2C). Contrary to our assumption, the presence of RC fraction in the bottom layer and DC in the top layer was puzzling, as DCs are considered as more dense organisms.

In particular, FIGS. 2A, 2B, 2C, and 2D illustrate the presence of DCs and RCs assessed in the TL and BLs of the renografin purified E. chaffeensis fractions. FIG. 2A provides a Western blot analysis performed with DnaK antibody which revealed higher levels of the protein expression in the BL derived bacterial fraction proteins (left half); similarly, ClpB protein expression was detected in the BL-derived proteins, but not in the top layer (right half). FIGS. 2B and 2C illustrate cell-free E. chaffeensis recovered from TL and BL assessed for reinfection of naïve Vero cells; infection was detected only with the fraction derived from the TL (FIG. 2B), but not in the BL (FIG. 2C). FIG. 2D illustrates the infectivity of fractionated Ehrlichia organisms from the TL and BL that was further confirmed by measuring the numbers of infected cells following incubation for 3 days following inoculation into naive HL60 cultures. Therefore to further confirm this observation, we performed transmission electron microscopy to detect the presence of DC and RC forms of E. chaffeensis in top and bottom layers, respectively (FIGS. 3A and 3B). Indeed, the pleomorphic and larger form of E. chaffeensis is evident primarily in the bottom layer, while the condensed form of the organisms were primarily observed in the top layer. Together, the extensive experimentation confirmed that E. chaffeensis DC form is present primarily in the PBS/25% gradient fraction interface (top layer), whereas the RC form is present in the 25/35% gradient fraction interface (bottom layer). In particular, FIGS. 3A and 3B illustrate transmission electron microscopy (TEM) analysis to define the E. chaffeensis organisms present in top and bottom layers of renografin fractions. TEM images of the top (FIG. 3A) and bottom (FIG. 3B) layers where prototypical small DCs and pleomorphic RCs of E. chaffeensis observed, respectively. Scale bars=1 μm.

We investigated if protein synthesis by the cell-free Ehrlichia in the axenic media has any pH preference and secondly if an altered pH may promote biosynthesis by the DC form (FIGS. 4A and 4B). As in the previous experiment, only RCs had the highest ³⁵S-Cys-Met incorporation and that the incorporation was not significantly different at varying pHs, while no major change was noted for the DC form of the bacterium. FIG. 4A and 4B illustrate the impact of pH variations assessed on the ³⁵S Cys-Met incorporation into E. chaffeensis cell-free organisms. In FIG. 4A, autoradiography imaging was performed to assess the impact of three different pH units; 5, 6 and 7. In the first lane, we included rifampin containing sample with media pH of 5.0 to serve as a negative control. FIG. 4B is similar to panel FIG. 4A, except that the scintillation counting data were presented.

We then assessed the impact of different energy sources for cell-free activity; the axenic medium supplemented with G6P, alpha ketoglutarate or sodium acetate supported the protein biosynthesis similarly for RC form of E. chaffeensis recovered from DH82 cultures although G6P appeared to be more favored but not significantly different as the carbon source (FIGS. 5A and 5B). We also tested the impact of the exclusion of a reducing agent (DTT) in the axenic medium for the protein biosynthesis. The absence of DTT had only a marginal negative effect. In FIGS. 5A and 5B, the axenic media was assessed using different carbon sources for the RC fraction. In FIG. 5A, autoradiography imaging was assessed with the axenic media containing G6P with DTT (G6P); without an energy source (no G6P); with G6P in the absence of DTT (G6P, no DTT); with alpha keto glutaric acid (α-KG) or sodium acetate (NaAceA) as the energy sources. The first lane included axenic media with G6P and CHL to serve as the negative control. FIG. 5B is similar to FIG. 5A, but scintillation counting data were presented.

Based on the incorporation data in the above-described experiments, we reasoned that only moderate protein biosysthesis has occurred by RCs. To validate these data, we compared protein profiles of total proteins resolved on an acrylamide gel before and after assessing by Western blot analysis using a murine polyclonal serum collected from E. chaffeensis infected mice (FIGS. 6A and 6B). Despite the presence of increased aboundance of a selected sub-group of proteins when RCs were incubated in the axenic medium containing different carbon sources, total protein increase was only moderate compared to that observed prior to incubating in the medium or in the axenic medium containing chloramphinicol (FIG. 6A). In FIG. 6A, silver stained PAGE in the presence of SDS resolved protein fractions were assessed for the protein abundance variations in RCs incubated in the axenic media with different carbon sources as in FIGS. 5A and 5B. Consistent with these data, Ehrlichia immunogenic proteins, assessed using polyclonal sera, were also moderately increased when the RCs were incubated with axenic medium using different carbon sources (FIG. 6B). FIG. 6B is similar to FIG. 6A, except that the resolved proteins were transferred to a nylon membrane and assessed by Western blot analysis using mouse polyclonal sera against E. chaffeensis.

We then tested if the axenic medium also promoted DNA synthesis (FIGS. 7A and 7B). ³H thymidine incorporation was assessed in the axenic medium simultaneously along with ³⁵S Cys-Met incorporation for incorporations into DNA and protein synthesis, respectively, for RC form of E. chaffeensis. This assay was carried out at different pH conditions in the medium ranging from pH 5 to 9. There was no major difference in the incorporation of ³H thymidine or ³⁵S Cys-Met when RCs were incubated at pHs 6-9, although the incorporation levels were the lowest for pH 5. Importantly, ³H thymidine incorporation in RCs DNA was consistent with that of the ³⁵S Cys-Met incorporation. FIG. 7A illustrates scintillation counting data for the ³⁵S Cys-Met assessed with G6P as the energy source. FIG. 7B is similar to FIG. 7A, except that the scintillation counting data for the ³H thymidine incorporation was assessed.

Discussion

Two major limitations of carrying out research on obligate bacterial pathogens, particularly on rickettsials belonging to the Anaplasmataceae family, are the lack of fully established methods for targeted mutagenesis and the inability to grow the bacteria in the absence of a host cell. While targeted mutagenesis methods aid in understanding the contributions of various genes involved in pathogenesis and in defining the genes critical for the pathogens' vector and vertebrate host cell-specific growth, the ability to grow the pathogens in a cell-free media can facilitate greatly in studies focused on understanding functions of various bacterial proteins without the influence of a host cell. Further, growth in cell free medium will aid in rapidly recovering mutant organisms and also to clonally purify mutants. Indeed, recent studies on C. burnetii demonstrated that significant progress could be made with the advent of fully established methods of mutagenesis and axenic growth. In an effort to address these two major deficiencies for the field of research on Anaplasmataceae family pathogens, such as those belonging to the genera Ehrlichia and Anaplasma, we recently described methods for creating stable targeted mutations to both disrupt and also restore the function of a disrupted gene in E. chaffeensis. In the present study, we focused on the second major challenge for the field; the development of axenic culture medium for E. chaffeensis. We present the first evidence in the axenic media that the protein biosynthesis and DNA synthesis are possible in a host cell-free culture media for E. chaffeensis organisms. We believe that the data described here are critical in moving the field forward in various fronts; 1) making the improvements to the axenic media growth method in promoting transition of replicating form to infectious form in E. chaffeensis and in other related Anaplasmataceae pathogens, 2) to aid in identifying and characterizing effector proteins involved in influencing the host, 3) in studying the potential interactions of the bacterial phagosome with mitochondria, host cytoplasmic proteins and nucleus, and 4) in facilitating the clonal purification of mutated organisms following creating insertion mutations by random and targeted mutagenesis methods.

Firstly, we presented a method for purification of E. chaffeensis DCs and RCs from host cells by employing renografin density gradient centrifugation. We discovered that the DC form of the bacterium fractioned at a lower concentration of renografin compared to the RC form. The presence of DCs and RCs within the gradient fractions was confirmed by three independent methods; ability to infect naive host cells, morphology, and by protein expression. Our studies demonstrate that axenic media supported protein synthesis only in the RCs of E. chaffeensis. Similarly, we presented evidence that the cell-free media supported the DNA synthesis of RCs. Axenic media-specific protein synthesis was further confirmed by inclusion of inhibitors; chloramphenicol or rifampin in the cell-free media.

Axenic media-specific protein synthesis in E. chaffeensis is similar to a prior study demonstrating the cell-free protein biosynthesis for C. trachomatis. While the C. trachomatis study doesn't address if the axenic media also supports the gross DNA synthesis, we reasoned that the media for this intra-cellular pathogen might have similarly supported a limited DNA synthesis. In particular, in the current study we presented evidence that the axenic media also supports the bacterial DNA synthesis. Despite protein and DNA synthesis shown in the absence of a host cell for RCs, our data suggest that the abundance of proteins and DNA made is limited. Total bacterial proteins resolved on a polyacrylamide gel and followed by staining with silver nitrate and Western blot analysis suggested that the bacterial replication was limited in the axenic media incubation experiments. We further investigated if variations in the pH of the media and varying energy sources may improve the protein biosynthesis. Despite G6P appears as the best energy source, we did not note any significant variations in the protein synthesis in the axenic media and nor did the altered media pH and carbon sources promoted protein synthesis in DC fraction of E. chaffeensis.

Two important goals to improve the axenic media for E. chaffeensis are; 1) to modify the media conditions, such as adding thymidine in the media cocktail, to promote the increased DNA and protein synthesis resulting the continued replication of the RC form and 2) to transform the RCs to DCs under axenic media conditions. These improvements may be possible if the axenic media growth is assessed with host cell derived purified E. chaffeensis RC-containing phagosomes in place of purified RCs (FIG. 8). Indeed, we performed the initial experiments with phagosomes of A. phagocytophilum and E. chaffeensis (FIGS. 9-15) and demonstrated that improvements can be made to the cell-free growth media. We reasoned that the phagosomal microenvironment might mimic closure to in vivo condition, although it may limit the number of bacterial replications. Axenic media growth may also be improved further if mitochondria are also added to the media-containing host cell-free RCs or RCs containing phagosomes (FIG. 8). In summary, the data presented in the manuscript represent a significant step forward in advancing the goal of developing axenic media growth of E. chaffeensis. We believe that the method needs improvement and that it can be adapted to other important pathogens belonging to Anaplasmataceae, such as other Ehrlichia species and Anaplasma species.

### EXAMPLE 2

This example uses the medium previously described for C. trachomatis, referred to as CIP-120, to evaluate the possibility of developing axenic culture methods for both E. chaffeensis RC-containing phagosomes and A. phagocytophilum RC-containing phagosomes in the absence of host cells.

Materials and Methods

Cell Lines and Cultivation of A. Phagocytophilum and E. Chaffeensis

The human promyelocytic cell lines HL-60 (ATCC CCL-240, Manassas, Va.) and A. phagocytophilum strain NCH-1-infected HL-60 was cultured in complete RPMI 1640 medium (Gibco/ThermoFisher Scientific) supplemented with 10% fetal bovine serum (Invitrogen/ThermoFisher Scientific, Waltham, Mass.) and 2 mM L-glutamine (Mediatech, Manassas, Va.), by following the protocols described elsewhere for Anaplasma phagocytophilum strain NCH-1. Cultivation of E. chaffeensis in HL60 cells in complete RPMI 1640 medium (Gibco/ThermoFisher Scientific) supplemented with 10% fetal bovine serum (Invitrogen/ThermoFisher Scientific, Waltham, Mass.) and 2 mM L-glutamine (Mediatech, Manassas, Va.) was performed by following the protocols described for Anaplasma phagocytophilum strain NCH-1. To prepare cell-free Anaplasmainocula, about 80-100% A. phagocytophilum-infected HL-60 cells in a T25 flask were harvested by centrifugation at 400×g for 10 minutes at 4° C. The pellets were resuspended in 5 ml of serum-free medium, and the cells were disrupted with glass beads by vortexing twice for 30 seconds. The cell debris and unbroken cells were removed by centrifugation at 200×g for 10 minutes at 4° C. The supernatant was passed through a 2.7 μm pore-size syringe filter (Whatman, Pittsburgh, Pa.). HL60 cells were incubated with host cell-free A. phagocytophilum (multiplicity of infection of 100:1, bacteria to host cell) for 120 minutes to allow for internalization. Non-ingested A. phagocytophilum were removed by washing with PBS, and the cells were incubated for 36 to 48 hours in a T150 flask. Similar infection protocol is followed when infecting by E. chaffeensis. The infected host cell cultures were harvested by centrifugation at 600×g for 5 minutes at 4° C. and used for purifying the host cell-free phagosomes, as outlined below.

Purification of Phagosomes

Purification of phagosomes from the A. phagocytophilum-infected HL-60 and E. chaffeensis-infected HL-60 was performed by subjecting to sugar density gradient centrifugation in combination with magnet assisted cell sorting (MACS) as described previously with some minor modifications. In brief, the infected HL60 cells were pelleted at 4° C. for 5 minutes at 350×g. The cells were washed twice with PBS and once with homogenization buffer (250 mM sucrose, 0.5 mM EGTA, 20 mM HEPES/KOH, pH 7.2). Cells were then resuspended in homogenization buffer with protease inhibitor cocktail (Sigma-Aldrich, St. Louis, Mo., USA). The cells were then homogenized at 4° C. using a 10 ml syringe with a 23^{½}-G needle; typically 10-15 strokes were used to disrupt the cells. Homogenization was carried out until approximately 90% of cells were disrupted without major breakage of nuclei, as monitored by light microscopy. Whole cells and nuclei were then pelleted in a 15 mL tube at 4° C. for 5 minutes at 300×g.

The resulting supernatant contained phagosomes, which was designated as the post-nuclear supernatant (PNS). The PNS was brought to a final concentration of 39% sucrose. The sucrose gradient was made by layering 1 mL of the PNS (39% sucrose) onto 2 mL of 55% sucrose layered onto 1 mL of 65% sucrose. We then layered 2 mL of 10% sucrose onto 2 mL of 25% sucrose solution onto the PNS. The phagosomes were isolated from the 55-65% interface using a 16g needle and not disturbing any other fraction. For further MACS separation, the crude phagosomes were incubated with rabbit Rab5 (1:1000) antibody for 1.5 hours, followed by incubation with MACS secondary goat anti-rabbit antibody (1:100, Miltenyi) for another 1.5 hours, and then loaded on a MACS LS separation column (Miltenyi) column in steps of 2 mL and washed with three times the input volume of HSMG buffer. The phagosomes were then eluted with 3 mL HSMG buffer HSMG buffer (20 mM HEPES, 250mM sucrose, 1.5mM MgCl2, 0.5 mM EGTA, pH 7.4) after removal of the magnet, aided by gentle pushing using the supplied plunger. The elution was placed into 10 mL of PBS (4° C.) and centrifuged at 40,000 g for 30 minutes at 4° C. The final purified pellets were resuspended in PBS for use in the cell free activity experiments.

Confocal Microscopy Analysis

The final purified phagosomes were plated onto 8-well culture chamber slide to adhere for 1 hour, and then incubated with Alexa Flour goat antirabbit 488 for 1 hour. The slides were washed with PBS and mounted with the mounting media containing DAPI. For infected HL60 cells staining, the infected HL60 cells were plated onto 8-well culture chamber slide and allowed to adhere for 1 hour in the 37° C. incubator. The cells were fixed with 4% formadehyde for 10 minutes at room temperature and permeabilized for 10 minutes. Subsequently, the cells were stained with rabbit Rab5 (1:1000) antibody overnight at 4° C. The antigen slides were washed with PBS to remove unbound primary antibody, and incubated with second antibody (Alexa Flour goat antirabbit 488) for 1 hour. The slides were washed with PBS and mounted with the mounting media containing DAPI. The slides were examined with a Zeiss LSM 700 laser scanning confocal microscopy (Carl Zeiss Optronics GmbH, Oberkochen, Germany).

Preparation of axenic medium: The axenic medium was prepared according to the previous study on C. trachomatis cultured in axenic medium, and the compositions and concentrations of each component in the axenic medium were listed in Table 1. Depending on the experiment carried out, the medium contained or excluded G6P or ATP to serve as carbon sources. Similarly, pH of the media is modified as per the experimental need.

Protein synthesis by ³⁵S-cysteine-methionine incorporation: Protein synthesis in cell-free purified phagosomes of A. phagocytophilum or E. chaffeensis was measured by incorporation of ³⁵5-Cys-Met (Perkin Elmer, Waltham, Mass.) as described previously. For normalization of bacterial total protein content, the suspension of the cell-free phagosomes were lysed in 1% (wt/vol) SDS for 5 minutes at 100° C. and the total protein concentration was determined using Protein Assay kit (Bio-Rad, Hercules, Calif.). Subsequently, the suspensions of cell-free phagosomes were equally split into micro-centrifuge tubes at the amount of 30 μg total protein. Partially opened micro-centrifuge tubes containing 500 μL of medium supplemented with 70 μCi of ³⁵S-Cys-Met were incubated 24 hours at 37° C. in a tri-gas incubator set to maintain 2.5% O₂. The cell-free organisms were pelleted at the end of incubation by centrifugation at 15,000×g for 15 minutes at 4° C., washed with K-36 buffer (0.05M K₂HPO₄, 0.05M KH₂PO₄, 0.1M KCl, 0.15M NaCl, pH7.0) twice, and disrupted by adding 30 μL of 2×SDS-PAGE sample buffer and by boiling for 5 minutes. Ten μL of lysate each was then transferred to a tube containing 5 mL of biosafe liquid II and used for quantification of ³⁵S-Cys-Met incorporation using the protocol 4 (³⁵S) in a liquid scintillation counter (TRI-CARB 2100TR, PerkinElmer, Waltham, Mass.). For visualizing the of radiolabel incorporation into phagosomes proteins, equal volumes of sample lysates were also separated in an SDS/PAGE and the gel was dried and exposed to an X-ray film. Similarly, cell-free growth experiments were carried out in the absence of added ³⁵S-Cys-Met, resolved on an SDS-PAGE gel and stained using silver nitrate staining kit (Pierce/ThermoFisher Scientific) as per the manufacturer's recommendations.

DNA synthesis by ³H-thymidine incorporation: Purified cell-free phagosomes were also assessed for incorporation of ³H-thymidine (Perkin Elmer, Waltham, Mass.) into the bacterial DNA simultaneously with the incorporation ³⁵5-Cys-Met into proteins. Briefly, A. phagocytophilum or E. chaffeensis cell-free phagosomes were incubated for 48 hours at 37° C. with 2.5% O₂ in micro-centrifuge tubes containing 500 μL of medium supplemented with 20 μCi of ³H-thymidine and 70 μCi of ³⁵5-Cys-Met. The phagosomes were pelleted at 15,000×g for 15 minutes at 4° C., washed with K-36 twice, and lysed in 30 μL of 2× SDS-PAGE sample buffer boiling for 5 minutes. 10 μL of lysate each was added into 5 mL of Biosafe liquid II (Grainger, Hartford, Conn.) and used for quantification of ³H-thymidine incorporation using the protocol 10 (³H) and ³⁵S-Cys-Met incorporation (the protocol 4, ³⁵S) by liquid scintillation counting (TRI-CARB 2100TR, PerkinElmer, Waltham, Mass.), respectively.

RNA Synthesis Assessed by Quantitative Real-Time RT-PCR

Quantitative real-time RT-PCR was employed to measure A. phagocytophilum or E. chaffeensis 16S rRNA expression. In brief, cultures of A. phagocytophilum or E. chaffeensis grown in several T150 flasks were used in recovering cell-free phagosomes form. The phagosomes organisms in triplicate microcentrifuge tubes were incubated for 0, 2, 6, 12, and 24 hours with 500 μL of axenic medium containing G6P and ATP at 37° C. with 2.5% O2. At the end of the specified incubation times, cells were recovered by centrifugation at 15,000×g for 10 minutes at 4° C. The bacterial pellets were then inactivated immediately in the TRI reagent solution, and then used to isolate total RNA by TRI reagent protocol (Sigma-Aldrich, St. Louis, Mo.). Final recovered RNA from each tube was resuspended in 25 μl of nuclease-free water, then DNase treated to remove residual genomic DNAs using RQ1 DNase (Thermo Fisher Scientific, Waltham, Mass.). RNA from each tube was diluted 1:1000 in nuclease-free water and 2 μl each was used in 25 μl reaction in performing Taq-Man probe-based real-time RT-PCR targeted to the E. chaffeensis 16S RNA as previously described. The RNA levels in each sample were expressed by Ct values. Variation among triplicates for each time point was calculated and presented with the respective standard deviations observed. Fold changes were calculated relative to the Ct values observed for the RNA recovered before incubation (0 hours) compared to different incubation times. The data were then assessed for statistical significance.

Sodium dodecyl sulfate polyacrylamide gel electrophoresis: Five μL of NuPAGE LDS sample buffer and 2 μL of NuPAGE reducing agent (Invitrogen/ThermoFisher Scientific) were added to each of 10 μL of sample solution following cell-free incubation experiments in the axenic medium, boiled for 5 minutes, and then loaded onto a Mini-PROTEAN Precast Bis-Tris 4% to 14% gels (Bio-rad, Hercules, Calif.) and subjected to electrophoresis (100 mA/gel for 60 minutes). The gels were then stained with Silver staining kit (Pierce/ThermoFisher Scientific) according to the manufacturer's recommendations.

Western blot analysis to assess protein synthesis: For detection of the DnaK and P28 proteins of E. chaffeensis or the Dnak of A. phagocytophilum, the above described electrophoresed proteins were transferred onto a nitrocellulose membrane (Thermo Fisher Scientific, Waltham, Mass.) by subjecting to electro-blotting using an electrophoretic transfer unit (Bio-Rad). Protein transfer buffer was prepared as per the manufacturer's instructions and used in the protein transfer protocols. Subsequently, E. chaffeensis DnaK and P28 expressions were assessed using the polyclonal rabbit antisera raised against a recombinant E. chaffeensis proteins for DnaK and P28, respectively. Secondary anti-rabbit antibody conjugated with horseradish peroxidase (Sigma-Aldrich, St. Louis, Mo., USA) and Super Signal West Pico Chemiluminescent Substrate (Thermo Fisher Scientific, Waltham, Mass., USA) were used for the signal detection, respectively.

Preparation of E. chaffeensis cultures for use in transmission electron microscopy: Purified E. chaffeensis phagosomes were resuspended in PBS and used in transmission electron microscopy analysis by following the methods described previously. Briefly, all centrifugation steps used in preparing the TEM samples were performed at 4° C. for 5 minutes at 200×g, unless otherwise specified. The cultures in PBS were fixed with 1 ml of Karnovsky's fixative containing 2% paraformaldehyde, 2.5% gluteraldehyde in 0.1 M cacodylate buffer (pH7.4) overnight. The cell-free E. chaffeensis organisms were then washed three times with 1 ml of 0.1 M cacodylate buffer and were incubated in 1 ml of 1% osmium tetraoxide in 0.1 M cacodylate buffer for one hour, washed three times using deionized water and then resuspended in 2% trypsin soy agar solution. Each sample was diced with a teflon coated razor blade and placed in a wheaton glass vials with 50% ethanol at room temperature for 15 minutes, then stained with 70% ethanol/uranyl acetate in the dark for one hour at room temperature. The bacterial suspension in soy agar was then subjected to dehydration process with an ethanol gradient of increased concentrations from 50% to 100%. All samples embedded in the soy agar-gluteraldehyde resin were subsequently transferred to silicon molds to allow for polymerization to be completed. All blocks were examined under a dissecting scope to identify a sample that was flush to the end of the block using an Ultracut E-Reichert-Jung ultramicrotome. Sections of 0.5 μm size were cut in the range of 75-90 nm, and transferred to Athene Thin Bar copper grids (Ted Pella, Redding, Calif.). The grids were stained with uranyl acetate in 70% ethanol followed by Reynold's lead citrate. The stained grids were examined under a Hitachi H-300 electron microscope (Hitachi High-Tech, San Jose, Calif.).

Results

FIG. 9 illustrates the phagosomes of the HL60 cell infected by A. phagocytophilum stained with DAPI (which stains the nucleus) and Rab 5 monoclonal antibody (which stains the borderline of phagosome). The Z-stack images are of the 4^{th} slice of the total 7 slices. The upper right photograph is presented under phase contrast. The upper right photograph is presented with DAPI staining. The lower left photograph is presented with Rab 5 Ab. Finally, the lower right photograph is combination of the upper right and lower left photographs superimposed on one another. As can be seen in the superimposed photo, the DAPI-stained A. phagocytophilum is located in the Rab 5 Ab-stained phagosome.

FIG. 10 illustrates the isolation of phagosomes from HL60 Cells infected by A. phagocytophilum with magnet assisted cell sorting. The left slide is under phase contrast, the middle slide is with DAPI staining, and the right slide is with Rab 5 Ab. Although a superimposed picture as provided in FIG. 9 is not included, the positioning of the DAPI-stained A. phagocytophilum and Rab 5 Ab-stained phagosomes indicates the same result as set forth in FIG. 9, namely that the Anaplasma is located in the Rab 5 Ab-stained phagosome.

FIG. 11 illustrates the isolation of phagosomes from HL60 cells infected by E. chaffeensis with magnet assisted cell sorting. The upper right photograph is presented under phase contrast. The upper right photograph is presented with DAPI staining. The lower left photograph is presented with Rab 5 Ab. Finally, the lower right photograph is combination of the upper right and lower left photographs superimposed on one another. Similar to FIG. 9, the DAPI-stained E. chaffeensis is located in the Rab 5 Ab-stained phagosome.

FIG. 12A illustrates the impact of energy sources on incorporation of ^{S}35 Cys-Met into A. phagocytophilum cell-free phagosomes. As expected, the lane with CHL showed no incorporation. In contrast, all 3 energy sources, G6P, ATP, and a combination of G6P and ATP all showed incorporation and evidence of protein synthesis.

FIG. 12B illustrates the impact of energy sources on incorporation of ^{S}35 Cys-Met into E. chaffeensis cell-free phagosomes. As expected, the lane with CHL showed no incorporation. In contrast, all 3 energy sources, G6P, ATP, and a combination of G6P and ATP all showed incorporation and evidence of protein synthesis.

FIG. 12C illustrates the impact of energy sources on incorporation of ^{S}35 Cys-Met into A. phagocytophilum cell-free phagosomes. As expected, the lane with CHL showed no incorporation. In contrast, all 3 energy sources, G6P, ATP, and a combination of G6P and ATP all showed incorporation and evidence of protein synthesis.

FIG. 12D illustrates the impact of energy sources on incorporation of ^{S}35 Cys-Met into E. chaffeensis cell-free phagosomes. As expected, the lane with CHL showed no incorporation. In contrast, all 3 energy sources, G6P, ATP, and a combination of G6P and ATP all showed incorporation and evidence of protein synthesis.

FIG. 13A illustrates the impact of pH variations on incorporation of ^{S}35 Cys-Met into A. phagocytophilum cell-free phagosomes. As can be seen, a pH between 5 and 8 all supported synthesis.

FIG. 13B illustrates the impact of pH variations on incorporation of ^{S}35 Cys-Met into E. chaffeensis cell-free phagosomes. As can be seen, a pH between 5 and 8 all supported synthesis.

FIG. 13C illustrates the impact of pH variations on incorporation of ^{S}35 Cys-Met into A. phagocytophilum cell-free phagosomes. As can be seen, a pH between 5 and 8 all supported synthesis.

FIG. 13D illustrates the impact of pH variations on incorporation of ^{S}35 Cys-Met into E. chaffeensis cell-free phagosomes. As can be seen, a pH between 5 and 8 all supported synthesis.

FIG. 14A illustrates DNA synthesis assessed simultaneously by measuring the 3H thymidine incorporation in the axenic media at varying pHs of the media in A. phagocytophilum cell-free phagosomes. As can be seen, a pH between 5 and 8 all supported DNA synthesis.

FIG. 14B illustrates DNA synthesis assessed simultaneously by measuring the 3H thymidine incorporation in the axenic media at varying pHs of the media in E. chaffeensis cell-free phagosome. As can be seen, a pH between 5 and 8 all supported DNA synthesis.

FIGS. 15A and 15B illustrate protein biosynthesis assessed by protein fractionation and Western blot analysis wherein silver stained SDS containing polyacrylamide gel-resolved protein fractions were assessed for the protein abundance variations in A. phagocytophilum cell-free phagosome. As can be seen, no synthesis occurs in the CHL-containing lanes while all other lanes provide evidence of synthesis at a pH between 5 and 8.

FIGS. 15C and 15D illustrate resultss similar to FIGS. 15A and 15B but wherein protein biosynthesis was assessed by Western blot analysis using mouse monoclonal Antibody DnaK and P28 against A. phagocytophilum. As is known, DnaK is present in both A. phagocytophilum and E. chaffeensis whereas P28 is only in E. chaffeensis.

1. A composition comprising:
a) intracellular phosphate buffer (IPB);
b) a carbon source;
c) FBS;
d) a mixture of amino acids; and
e) at least one further component selected from the group consisting of glucose 6-phosphate (G6P), ATP, DTT, GTP, UTP, CTP, and any combination thereof.

2. The composition of claim 1 further comprising phagosomes and/or mitochondria.

3. (canceled)

4. The composition of claim 1, further comprising a bacterium from the family Anaplasmataceae.

5. The composition of claim 4, wherein the bacterium is selected from the group consisting of Ehrlichia chaffeensis, Ehrlichia ewingii, Ehrlichia canis, Ehrlichia ruminantium, Ehrlichia muris euclarancis, Anaplasma marginale, Anaplasma phagocytophilum, and any combination thereof.

6. The composition of claim 1, wherein said IPB comprises a mixture of at least one phosphate, at least one gluconate, and at least one chloride.

7. The composition of claim 6, wherein said at least one phosphate is selected from the group consisting of potassium phosphate, sodium phosphate, and any combination thereof, and/or wherein said at least one chloride is selected from the group consisting of potassium chloride, magnesium chloride, and any combination thereof.

8. The composition of claim 6, wherein said at least one phosphate is included at a final concentration of at least 1 mM and/or wherein said gluconate is included at a final concentration between 70 mM and 150 mM, and/or wherein said at least one chloride is included in the medium at a final concentration between 0.5 mM and 12 mM.

9. The composition of claim 6, wherein said phosphate comprises a mixture of potassium phosphate and sodium phosphate.

10-12. (canceled)

13. The composition of claim 1, wherein said carbon source is alpha ketoglutarate and/or sodium acetate.

14. The composition of claim 1, wherein said carbon source is included at a concentration of at least 0.1 mM.

15. The composition of claim 1, wherein said mixture of amino acids includes at least 5 different amino acids.

16. The composition of claim 1, wherein any two of GTP, UTP, and CTP are present.

17. The composition of claim 1, wherein when ATP is present, a quantity of DTT is also included.

18. A method of cell free protein synthesis of a bacterium from the family Anaplasmataceae comprising the step of contacting the bacteria with the composition of claim 1 for a period of time sufficient to permit protein synthesis.

19. The method of claim 18, wherein the time period is at least 12 hours and/or wherein said bacteria are surrounded by said composition and/or wherein said composition has a pH between 5 and 9 and/or wherein the cell free protein synthesis is performed at a temperature between 24° C. to 40° C.

20-23. (canceled)

24. The method of claim 18, wherein said bacterium is selected from the group consisting of Ehrlichia chaffeensis, Ehrlichia ewingii, Ehrlichia canis, Ehrlichia ruminantium, Ehrlichia muris euclarancis, Anaplasma marginale, Anaplasma phagocytophilum, and any combination thereof.

25. A method of cell free nucleic acid synthesis of a bacteria from the family Anaplasmataceae comprising the step of contacting the bacteria with the composition of claim 1 for a period of time sufficient to permit nucleic acid synthesis.

26. The method of claim 25, wherein the time period is at least 12 hours and/or wherein said bacteria are surrounded by said composition and/or wherein said composition has a pH between 5 and 9 and/or wherein the cell free protein synthesis is performed at a temperature between 24° C. to 40° C.

27-28. (canceled)

29. The method of claim 25, wherein the time period is between 1 day and 7 days.

30. (canceled)

31. The method of claim 25, wherein said bacterium is selected from the group consisting of Ehrlichia chaffeensis, Ehrlichia ewingii, Ehrlichia canis, Ehrlichia ruminantium, Ehrlichia muris euclarancis, Anaplasma marginale, Anaplasma phagocytophilum, and any combination thereof.
